(19) United States
(12) Patent Application Publication
(10) Pub. No.: US 2015/0252340 A1
(43) Pub. Date: Sep. 10, 2015

(54) COMPOSITIONS AND METHODS OF US

(71) Applicant: DANISCO US INC., Palo Alto, CA (US)

(72) Inventors: Benjamin S. Bower, Newark, CA (US); Meredith K. Fujdala, San Jose, CA (US)

(73) Assignee: DANISCO US INC., Palo Alto, CA (US)

(21) Appl. No.: 14/437,141

(22) PCT Filed: Oct. 30, 2013
(86) PCT No.: PCT/US2013/067419

§ 371 Date: Apr. 20, 2015

Related U.S. Application Data
(60) Provisional application No. 61/720,732, filed on Oct. 31, 2012.

(51) Int. Cl. C12N 9/24; C12P 19/02

(52) U.S. Cl. CPC C12N 9/2402; C12P 19/02; C12Y 302/01021

(57) ABSTRACT

The present compositions and methods relate to a beta-glucosidase from Aspergillus terreus, polynucleotides encoding the beta-glucosidase, and methods of make and/or use thereof. Formulations containing the beta-glucosidase are suitable for use in hydrolyzing lignocellulosic biomass substrates.

### PRIORITY

The present application claims priority to U.S. Provisional Application Ser. No. 61/720,732, filed on Oct. 31, 2012, which is hereby incorporated by reference in its entirety.

### TECHNICAL FIELD

The present compositions and methods relate to a beta-glucosidase polypeptide obtainable from Aspergillus terreus, polynucleotides encoding the beta-glucosidase polypeptide, and methods of making and using thereof. Formulations and compositions comprising the beta-glucosidase polypeptide are useful for degrading or hydrolyzing lignocellulosic biomass.

### DESCRIPTION OF THE BACKGROUND

Cellulose and hemicellulose are the most abundant plant materials produced by photosynthesis. They can be degraded and used as an energy source by numerous microorganisms (e.g., bacteria, yeast and fungi) that produce extracellular enzymes capable of hydrolysis of the polymeric substrates to monomeric sugars (Aro et al., (2001) J. Biol. Chem., 276: 24309-24314). As the limits of non-renewable resources approach, the potential of cellulose to become a major renewable energy resource is enormous (Krishna et al., (2001) Bioresource Tech., 77: 193-196). The effective utilization of cellulose through biological processes is one approach to overcoming the shortage of foods, feeds, and fuels (Ohmiya et al., (1997) Biotechnol. Gen. Engineer Rev., 14: 365-414).

Cellulases are enzymes that hydrolyze cellulose (comprising beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”) (Knowles et al., (1987) TIBTECH 5: 255-261; and Schulein, (1988) Methods Enzymol., 160: 234-243). Endoglucanases act mainly on the amorphous parts of the cellulose fiber, whereas cellobiohydrolases are also able to degrade crystalline cellulose (Nevalainen and Penttila, (1995) Mycota, 303-319). Thus, the presence of a cellobiohydrolase in a cellulase system is required for efficient solubilization of crystalline cellulose (Suurnakki et al., (2000) Cellulose, 7: 189-209). Beta-glucosidase acts to liberate D-glucose units from cellobiose, cello-oligosaccharides, and other glucosides (Freer, (1993) J. Biol. Chem., 268: 9337-9342).

Cellulases are known to be produced by a large number of bacteria, yeast and fungi. Certain fungi produce a complete cellulase system capable of degrading crystalline forms of cellulose. These fungi can be fermented to produce suites of cellulases or cellulase mixtures. The same fungi and other fungi can also be engineered to produce or overproduce certain cellulases, resulting in mixtures of cellulases that comprise different types or proportions of cellulases. The fungi can also be engineered such that they produce in large quantities via fermentation the various cellulases. Filamentous fungi play a special role since many yeast, such as Saccharomyces cerevisiae, lack the ability to hydrolyze cellulose in their native state (see, e.g., Wood et al., (1988) Methods in Enzymology, 160: 87-116).

The fungal cellulase classifications of CBH, EG and BG can be further expanded to include multiple components within each classification. For example, multiple CBHs, EGs and BGs have been isolated from a variety of fungal sources including Trichoderma reesei (also referred to as Hypocrea jecorina), which contains known genes for two CBHs, i.e., CBH I (“CBH1”) and CBH II (“CBH2”), at least eight EGs, i.e., EG I, EG II, EG III, EGIV, EGV, EGVI, EGVII and EGVIII, and at least five BGs, i.e., BG1, BG2, BG3, BG4, BG5 and BG7 (Foreman et al. (2003), J. Biol. Chem. 278(34):31988-31997). EGIV, EGVI and EGVIII also have xyloglucanase activity.

In order to efficiently convert crystalline cellulose to glucose the complete cellulase system comprising components from each of the CBH, EG and BG classifications is required, with isolated components less effective in hydrolyzing crystalline cellulose (Filho et al., (1996) Can. J. Microbiol., 42:1-5). Endo-1,4-beta-glucanases (EG) and exo-cellobiohydrolases (CBH) catalyze the hydrolysis of cellulose to cellooligosaccharides (cellobiose as a main product), while beta-glucosidases (BGL) convert the oligosaccharides to glucose. A synergistic relationship has been observed between cellulase components from different classifications. In particular, the EG-type cellulases and CBH-type cellulases synergistically interact to efficiently degrade cellulose. The beta-glucosidases serves the important role of liberating glucose from the cellooligosaccharides such as cellobiose, which is toxic to the microorganisms, such as, for example, yeasts, that are used to ferment the sugars into ethanol; and which is also inhibitory to the activities of endoglucanases and cellobiohydrolases, rendering them ineffective at further hydrolyzing the crystalline cellulose.

In view of the important role played by beta-glucosidases in the degradation or conversion of cellulosic materials, discovery, characterization, preparation, and application of beta-glucosidase homologs with improved efficacy or capability to hydrolyze cellulosic feedstock is desirable and advantageous.

### SUMMARY OF THE INVENTION

Beta-glucosidase Obtainable from Aspergillus terreus and their Use

Enzymatic hydrolysis of cellulose remains one of the main limiting steps of the biological production from lignocellulosic biomass feedstock of a material, which may be cellulosic sugars and/or downstream products. Beta-glucosidases play the important role of catalyzing the last step of that process, releasing glucose from the inhibitory cellobiose, and therefore its activity and efficacy directly contributes to the overall efficacy of enzymatic lignocellulosic biomass conversion, and consequently to the cost in use of the enzyme solution. Accordingly there is great interest in finding, making and using new and more effective beta-glucosidases.

While a number of beta-glucosidases are known, including the beta-glucosidases Bgl1, Bgl3, Bgl5, Bgl7, etc, from Trichoderma reesei or Hyprocrea jecorina (Korotkova O.G. et al., (2009) Biochemistry 74:569-577; Chauve, M. et al., (2010) Biotechnol. Biofuels 3:3-3), the beta-glucosidases from Humicola grisea var. thermoidea (Nascimento, C.V. et al., (2010) J. Microbiol. 48, 53-62); from Sporotrichum pulverulentum, Deshpande V. et al., (1988) Methods Enzymol., 160:415-424); of Aspergillus oryzae(Fukuda T. et al., (2007) Appl. Microbiol. Biotechnol. 76:1027-1033, from Talaromyces thermophilusCBS 236.58 (Nakkharat P. et al., (2006) J. Biotechnol., 123:304-313), from Talaromyces emersonii(Murray P., et al., (2004) Protein Expr. Purif. 38:248-257), so far the Trichoderma reesei beta-glucosidase Bgl1 and the Aspergillus niger beta-glucosidase SP188 are deemed benchmark beta-glucosidases against which the activities and performance of other beta-glucosidases are evaluated. It has been reported that Trichoderma reesei Bgl1 has higher specific activity than Aspergillus niger beta-glucosidase SP188, but the former can be poorly secreted, while the latter is more sensitive to glucose inhibition (Chauve, M. et al., (2010) Biotechnol. Biofuels, 3(1):3).

One aspect of the present compositions and methods is the application or use of a highly active beta-glucosidase isolated from the fungal species Aspergillus terreus strain NIH2624, to hydrolyze a lignocellulosic biomass substrate. The genome of Aspergillus terreus strain NIH2624 has been annotated in 2005, and the herein described sequence of SEQ ID NO:2 was published by National Center for Biotechnology Information, U.S. National Library of Medicine (NCBI) with the Accession No. XP_{—}001212225.1, and designated to be a beta-glucosidase I precursor. The enzyme has previously been expressed in a transgenic dicot plant (e.g., a soybean plant) to enhance the seed's capability to produce a desired protein to as much as 5% of the dry weight of the seed. See, e.g., WO02009158716. In that case, the beta-glucosidase polypeptide of Aspergillus terreus was expressed by the transgenic plant as an enhancer for protein storage. In another example, it has been described in WO02009108941, that the beta-glucosidase polypeptide of Aspergillus terreus may be expressed in a plant such that the plant extract can be combined with a bacterial extract in order to help the plant release fermentable sugars. On the other hand, the beta-glucosidase of Aspergillus terreus has not previously been expressed by an engineered microorganism. Nor has it been co-expressed with one or more cellulase genes and/or one or more hemicellulase genes. Expression in suitable microorganisms, which have, through many years of development, become highly effective and efficient producers of heterologous proteins and enzymes, with the aid of an arsenal of genetic tools, makes it possible to express these useful beta-glucosidases in substantially larger amounts than when they are expressed endogenously in an unengineered microorganism, or when they are expressed in plants. Enzymes classified as beta-glucosidases are diverse not only in their origins but also in their activities on lignocellulosic substrates, although most if not all beta-glucosidases can catalyze cellobiose hydrolysis under suitable conditions. For example, some are active on not only cellobiose, but also on longer-chain oligosaccharides, whereas others are more exclusively active only on cellobiose. Even for those beta-glucosidases that have similar substrate preferences, some have enzyme kinetics profiles that make them more catalytically active and efficient, and accordingly more useful in industrial applications where the enzymatically catalyzed hydrolysis cannot afford to take longer than a few days at most. Furthermore, no fermenting or ethanologen microorganism capable of converting cellulosic sugars obtained from enzymatic hydrolysis of lignocellulosic biomass has been engineered to express a beta-glucosidase from Aspergillus terreus, such as an Ate3C polypeptide herein. Expression of beta-glucosidases in ethanologen microorganisms provides an important opportunity to further liberating D-glucose from the remaining cellobiose that are not completely converted by the enzyme saccharification, where the D-glucose thus produced can be immediately consumed or fermented just in time by the ethanologen.

An aspect of the present composition and methods pertains to beta-glucosidase polypeptides of glycosyl hydrolase family 3 derived from Aspergillus terreus, referred to herein as “Ate3C” or “Ate3C polypeptides,” nucleic acids encoding the same, compositions comprising the same, and methods of producing and applying the beta-glucosidase polypeptides and compositions comprising thereof in hydrolyzing or converting lignocellulosic biomass into soluble, fermentable sugars. Such fermentable sugars can then be converted into cellulosic ethanol, fuels, and other biochemicals and useful products. In certain embodiments, the Ate3C beta-glucosidase polypeptides have higher beta-gluclosidase activity and/or exhibits an increased capacity to hydrolyze a given lignocellulosic biomass substrate as compared to the benchmark Trichderma reesei Bgl1, which is a known, high fidelity beta-glucosidase. (Chauve, M. et al., (2010) Biotechnol. Biofuels, 3(1):3).

In some embodiments, an Ate3C polypeptide is applied together with, or in the presence of, one or more other cellulases in an enzyme composition to hydrolyze or breakdown a suitable biomass substrate. The one or more other cellulases may be, for example, other beta-glucosidases, cellobiohydrolases, and/or endoglucanases. For example, the enzyme composition may comprise an Ate3C polypeptide, a cellobiohydrolase, and an endoglucanase. In some embodiments, the Ate3C polypeptide is applied together with, or in the presence of, one or more hemicellulases in an enzyme composition. The one or more hemicellulases may be, for example, xylanases, beta-xylosidases, and/or L-arabinofuranosidases. In further embodiments, the Ate3C polypeptide is applied together with, or in the presence of, one or more cellulases and one or more hemicellulases in an enzyme composition. For example, the enzyme composition comprises an Ate3C polypeptide, no or one or two other beta-glucosidases, one or more cellobiohydrolases, one or more endoglucanases; optionally no or one or more xylanases, no or one or more beta-xylosidases, and no or one or more L-arabinofuranosidases.

In certain embodiments, an Ate3C polypeptide, or a composition comprising the Ate3C polypeptide is applied to a lignocellulosic biomass substrate or a partially hydrolyzed lignocellulosic biomass substrate in the presence of an ethanologen microbe, which is capable of metabolizing the soluble fermentable sugars produced by the enzymatic hydrolysis of the lignocellulosic biomass substrate, and converting such sugars into ethanol, biochemicals or other useful materials. Such a process may be a strictly sequential process whereby the hydrolysis step occurs before the fermentation step. Such a process may, alternatively, be a hybrid process, whereby the hydrolysis step starts first but for a period overlaps the fermentation step, which starts later. Such a process may, in a further alternative, be a simultaneous hydrolysis and fermentation process, whereby the enzymatic hydrolysis of the biomass substrate occurs while the sugars produced from the enzymatic hydrolysis are fermented by the ethanologen.

The Ate3C polypeptide, for example, may be a part of an enzyme composition, contributing to the enzymatic hydrolysis process and to the liberation of D-glucose from oligosaccharides such as cellobiose. In certain embodiments, the Ate3C polypeptide may be genetically engineered to express in an ethanologen, such that the ethanologen microbe expresses and/or secrets such a beta-glucosidase activity. Moreover, the Ate3C polypeptide may be a part of the hydrolysis enzyme composition while at the same time also expressed and/or secreted by the ethanologen, whereby the soluble fermentable sugars produced by the hydrolysis of the lignocellulosic biomass substrate using the hydrolysis enzyme composition is metabolized and/or converted into ethanol by an ethanologen microbe that also expresses and/or secrets the Ate3C polypeptide. The hydrolysis enzyme composition can comprise the Ate3C polypeptide in addition to one or more other cellulases and/or one or more hemicellulases. The ethanologen can be engineered such that it expresses the Ate3C polypeptide, one or more other cellulases, one or more other hemicellulases, or a combination of these enzymes. One or more of the beta-glucosidases may be in the hydrolysis enzyme composition and expressed and/or secreted by the ethanologen. For example, the hydrolysis of the lignocellulosic biomass substrate may be achieved using an enzyme composition comprising an Ate3C polypeptide, and the sugars produced from the hydrolysis can then be fermented with a microorganism engineered to express and/or secret Ate3C polypeptide. Alternatively, an enzyme composition comprising a first beta-glucosidase participates in the hydrolysis step and a second beta-glucosidase, which is different from the first beta-glucosidase, is expressed and/or secreted by the ethanologen. For example, the hydrolysis of the lignocellulosic biomass substrate may be achieved using a hydrolysis enzyme composition comprising Trichoderma reesei Bgl1, and the fermentable sugars produced from hydrolysis are fermented by an ethanologen microorganism expressing and/or secreting an Ate3C polypeptide, or vice versa.

As demonstrated herein, Ate3C polypeptides and compositions comprising Ate3C polypeptides have improved efficacy at conditions under which saccharification and degradation of lignocellulosic biomass take place. The improved efficacy of an enzyme composition comprising an Ate3C polypeptide is shown when its performance of hydrolyzing a given biomass substrate is compared to that of an otherwise comparable enzyme composition comprising Bgl1 of Trichoderma reesei.

In certain embodiments, the improved or increased beta-glucosidase activity is reflected in an improved or increased cellobiase activity of the Ate3C polypeptides, which is measured using cellobiose as substrate, for example, at a temperature of about 30° C. to about 65° C. (e.g., about 35° C. to about 60° C., about 40° C. to about 55° C., about 45° C. to about 55° C., about 48° C. to about 52° C., about 40° C., about 45° C., about 50° C., about 55° C., etc). In some embodiments, the improved beta-glucosidase activity of an Ate3C polypeptide as compared to that of Trichoderma reesei Bgl1, is observed when the beta-glucosidase polypeptides are used to hydrolyze a phosphoric acid swollen cellulose (PASC), for example, a thus pretreated Avicel pretreated using an adapted protocol of Walseth, TAPPI 1971, 35:228 and Wood, Biochem. J. 1971, 121:353-362. In some embodiments, the improved beta-glucosidase activity of an Ate3C polypeptide as compared to that of Trichoderma reesei Bgl1, is observed when the beta-glucosidase polypeptides are used to hydrolyze a dilute ammonia pretreated corn stover, for example, one described in International Published Patent Applications: WO2006110891, WO2006110899, WO2006110900, WO2006110901, and WO2006110902; U.S. Pat. Nos. 7,998,713, 7,932,063.

In some aspects, an Ate3C polypeptide and/or as it is applied in an enzyme composition or in a method to hydrolyze a lignocellulosic biomass substrate is (a) derived from, obtainable from, or produced by Aspergillus terreus strain NIH2624; (b) a recombinant polypeptide comprising an amino acid sequence that is at least 85% (e.g., at least 85%, 90%, 91%, 92%, 93%, 94%, 95%, 96%, 97%, 98%, 99%, or 100%) identical to the amino acid sequence of SEQ ID NO:2; (c) a recombinant polypeptide comprising an amino acid sequence that is at least 85% (e.g., at least 85%, 90%, 91%, 92%, 93%, 94%, 95%, 96%, 97%, 98%, 99%, or 100%) identical to the catalytic domain of SEQ ID NO:2, namely amino acid residues 20 to 861; (d) a recombinant polypeptide comprising an amino acid sequence that is at least 85% (e.g., at least 85%, 90%, 91%, 92%, 93%, 94%, 95%, 96%, 97%, 98%, 99%, or 100%) identical to the mature form of amino acid sequence of SEQ ID NO:3, namely amino acid residues 20-861 of SEQ ID NO:2; or (e) a fragment of (a), (b), (c) or (d) having beta-glucosidase activity. In certain embodiments, it is provided a variant polypeptide having beta-glucosidase activity, which comprises a substitution, a deletion and/or an insertion of one or more amino acid residues of SEQ ID NO:2.

In some aspects, an Ate3C polypeptide and/or as it is applied in an enzyme composition or in a method to hydrolyze a lignocellulosic biomass substrate is (a) a polypeptide encoded by a nucleic acid sequence that is at least 85% (e.g., at least 85%, 90%, 91%, 92%, 93%, 94%, 95%, 96%, 97%, 98%, 99% or 100%) sequence identity to SEQ ID NO:1, or (b) one that hybridizes under medium stringency conditions, high stringency conditions or very high stringency conditions to SEQ ID NO:1 or to a subsequence of SEQ ID NO:1 of at least 100 contiguous nucleotides, or to the complementary sequence thereof, wherein the polypeptide has beta-glucosidase activity. In some embodiments, an Ate3C polypeptide and/or as it is applied in a composition or in a method to hydrolyze a lignocellulosic biomass substrate is one that, due to the degeneracy of the genetic code, does not hybridize under medium stringency conditions, high stringency conditions or very high stringency conditions to SEQ ID NO:1 or to a subsequence of SEQ ID NO:1 of at least 100 contiguous nucleotide, but nevertheless encodes a polypeptide having beta-glucosidase activity and comprising an amino acid sequence that is at least 85% (e.g., at least 85%, 90%, 91%, 92%, 93%, 94%, 95%, 96%, 97%, 98%, 99% or 100%) identical to that of SEQ ID NO:2 or to the mature beta-glucosidase sequence of SEQ ID NO:3. The nucleic acid sequences can be synthetic, and is not necessarily derived from Aspergillus terreus, but the nucleic acid sequence encodes a polypeptide having beta-glucosidase activity and comprises an amino acid sequence that is least 85% identical to SEQ ID NO:2 or to SEQ ID NO:3.

In some preferred embodiments, the Ate3C polypeptide or the composition comprising the Ate3C polypeptide has improved beta-glucosidase activity, as compared to that of the wild type Trichoderma reesei Bgl1 (of SEQ ID NO: 4), or the enzyme composition comprising the Trichoderma reesei Bgl1. In certain embodiments, the cellulase activity of the Ate3C polypeptide of the compositions and methods herein, as measured using a Chloro-nitro-phenyl-glucoside (CNPG) hydrolysis assay, is about 20% to about 60% (e.g., about 20% to about 55%, about 30% to about 55%, about 40% to about 60%, about 45% to about 55%) of that of Trichoderma reesei Bgl1. The CNPG assay is described in Example 2B herein. In some embodiments, the Ate3C polypeptide or the composition comprising the Ate3C polypeptide has improved beta-glucosidase activity, as compared to that of the wild type Aspergillus niger B-glu, or the enzyme composition comprising Aspergillus niger B-glu. In some embodiments, the cellulase activity of Ate3C polypeptide of the compositions and methods herein, as measured using a CNPG hydrolysis assay, is at least double, at least about 3 times, at least about 4 times, at least about 5 times higher than that of Aspergillus niger B-glu.

For example, the beta-glucosidase activity of the Ate3C polypeptide of the compositions and methods herein, as measured using a cellobiose hydrolysis assay, is at least about 5% higher (e.g., at least about 5% higher, at least about 10% higher, at least about 15% higher, at least about 20% higher, at least about 25% higher, at least about 30% higher, at least about 40% higher, at least about 50% higher, at least about 75% higher, at least about 100% higher, or even at least about 125% higher, such as, for example, at least about 150% higher) than that of the Trichoderma reesei Bgl1. The cellobiose hydrolysis assay is described in Example 2C herein. In some embodiments, the beta-glucosidase activity of the Ate3C polypeptide of the compositions and methods herein, as measured using a cellobiose hydrolysis assay (of Example 2C herein), is about half or less, is about ⅓ or less, is about ¼ or less than that of Aspergillus niger B-glu.

In some embodiments, the Ate3C polypeptides of the compositions and methods herein have improved cellobiose hydrolysis activity but reduced capacity to hydrolyze chloro-nitro-phenyl-glucoside (CNPG). For example, the Ate3C polypeptides of the composition and methods herein may have at least 20% higher “cellobiase” activity (i.e., measuring the capacity to hydrolyze cellobiose) but at least 20% lower activity hydrolyzing CNPG, as compared to Trichoderma reesei Bgl1. In some embodiments, the Ate3C polypeptides of the compositions and methods herein have less (for example, about ½ or less, about ⅓ or less, about ¼ or less) cellobiase activity but increased capacity to hydrolyze chloronitro-phenyl-glucoside (CNPG) (for example, by at least double, at least about 3 times, at least about 4 times), as compared to Aspergillus niger B-glu.

In some embodiments, the recombinant Ate3C polypeptide, as compared to the Trichoderma reesei Bgl1, has about 2-fold, about 3-fold, about 4-fold, or even about 5-fold lower hydrolysis activity ratio over CNPG/cellobiose. In some embodiments, the Ate3C polypeptide, as compared to the Aspergillus niger B-glu, has about 2-fold, about 3-fold, about 4-fold, about 5-fold, or even about 6-fold higher relative hydrolysis activity ratio over CNPG/cellobiose.

In certain aspects, the Ate3C polypeptides and the compositions comprising the Ate3C polypeptides of the invention have improved performance hydrolyzing lignocellulosic biomass substrates, as compared to that of the wild type Trichoderma reesei Bgl1 (of SEQ ID NO:4). In some embodiments, the improved hydrolysis performance of Ate3C polypeptides or compositions comprising Ate3C polypeptides is observable by the production of a greater amount of glucose from a given lignocellulosic biomass substrate, pretreated in a certain way, as compared to the level of glucose produced by Trichoderma reesei Bgl1 or an identical enzyme composition comprising Trichoderma reesei Bgl1 from the same biomass pretreated the same way, under the same saccharification conditions. For example, the amount of glucose produced by the Ate3C polypeptides or by the enzyme compositions comprising the Ate3C polypeptides is at least about 5% (e.g., at least about 5%, at least about 10%, at least about 15%, at least about 20%, at least about 25%, at least about 30%, at least about 35%, at least about 40%, at least about 45%, or even at least about 50%) greater than the amount produced by the Trichoderma reesei Bgl1 or an otherwise identical enzyme composition comprising the Trichoderma reesei Bgl1 (rather than an Ate3C polypeptide), when 0-10 mg (e.g., about 1 mg, about 2 mg, about 3 mg, about 4 mg, about 5 mg, about 6 mg, about 7 mg, about 8 mg, about 9 mg, about 10 mg) of beta-glucosidase (an Ate3C polypeptide or Trichoderma reesei Bgl1) is used to hydrolyze 1 g glucan in the biomass substrate.

In some aspects, the improved hydrolysis performance of Ate3C polypeptides or compositions comprising Ate3C polypeptides is observable by the production of an equal or reduced amount of total sugars from a given lignocellulosic biomass substrate pretreated in a certain way, as compared to the level of total sugars produced by Trichoderma reesei Bgl1 or an otherwise identical enzyme composition comprising Trichoderma reesei Bgl1 from the same biomass pretreated the same way, under the same saccharification conditions. For example, the amount of total sugars produced by the Ate3C polypeptides or the enzyme compositions comprising the Ate3C polypeptides is the same or at least about 5% (e.g., at least about 5%, at least about 10%, at least about 15%, at least about 20%, at least about 25%, at least about 30%, at least about 35%, at least about 40%, at least about 45%, or even at least about 50%) less than that the amount produced by Trichoderma reesei Bgl1 or an otherwise identical enzyme composition comprising Trichoderma reesei Bgl1 (rather than an Ate3C polypeptide), when 0.1 mg beta-glucosidase (an Ate3C polypeptide or Trichoderma reesei Bgl1) is used to hydrolyze 1 g glucan in the biomass substrate.

In further aspects, the improved hydrolysis performance of Ate3C polypeptides and compositions comprising Ate3C polypeptides is observable by an increased amount of glucose and an equal and reduced amount of total sugars produced from hydrolyzing a given lignocellulosic biomass substrate pretreated in a certain way, as compared to the amount of glucose and amount of total sugars produced by Trichoderma reesei Bgl1 or an otherwise identical composition comprising Trichoderma reesei Bgl1 from the same biomass pretreated the same way under the same saccharification conditions. For example, the amount of glucose produced by the Ate3C polypeptides or the compositions comprising the Ate3C polypeptides is at least about 5% (e.g., at least about 5%, at least about 10%, at least about 15%, at least about 20%, at least about 25%, at least about 30%, at least about 35%, at least about 40%, at least about 45%, or even at least about 50%) greater than the amount produced by the Trichoderma reesei Bgl1 or by an otherwise identical enzyme composition comprising Trichoderma reesei Bgl1, whereas the amount of total sugars produced by the Ate3C polypeptides or the compositions comprising the Ate3C polypeptides is the same or about 5% (e.g., at least about 5%, at least about 10%, at least about 15%, at least about 20%, at least about 25%, at least about 30%, at least about 35%, at least about 40%, at least about 45%, or even at least about 50%) less than the amount produced by the Trichoderma reesei Bgl1 or by an otherwise identical enzyme composition composition comprising Trichoderma reesei Bgl1, when 0-10 mg (e.g., about 1 mg, about 2 mg, about 3 mg, about 4 mg, about 5 mg, about 6 mg, about 7 mg, about 8 mg, about 9 mg, about 10 mg) of beta-glucosidase (an Ate3C polypeptide or Trichoderma reesei Bgl1) is used to hydrolyze 1 g glucan in the biomass substrate PASC.

Aspects of the present compositions and methods include a composition comprising a recombinant Ate3C polypeptide as detailed above and a lignocellulosic biomass. Suitable lignocellulosic biomass may be, for example, derived from an agricultural crop, a byproduct of a food or feed production, a lignocellulosic waste product, a plant residue, including, for example, a grass residue, or a waste paper or waste paper product. In certain embodiments, the lignocellulosic biomass has been subject to one or more pretreatment steps in order to render xylan, hemicelluloses, cellulose and/or lignin material more accessible or susceptible to enzymes and thus more amendable to enzymatic hydrolysis. A suitable pretreatment method may be, for example, subjecting biomass material to a catalyst comprising a dilute solution of a strong acid and a metal salt in a reactor. See, e.g., U.S. Pat. Nos. 6,660,506, 6,423,145. Alternatively, a suitable pretreatment may be, for example, a multi-stepped process as described in U.S. Pat. No. 5,536,325. In certain embodiments, the biomass material may be subject to one or more stages of dilute acid hydrolysis using about 0.4% to about 2% of a strong acid, in accordance with the disclosures of U.S. Pat. No. 6,409,841. Further embodiments of pretreatment methods may include those described in, for example, U.S. Pat. No. 5,705,369; in Gould, (1984) Biotech. & Bioengr., 26:46-52; in Teixeira et al., (1999) Appl. Biochem & Biotech., 77-79:19-34; in International Published Patent Application WO2004/081185; or in U.S. Patent Publication No. 20070031918, or International Published Patent Application WO06110901.

The present invention also pertains to isolated polynucleotides encoding polypeptides having beta-glucosidase activity, wherein the isolated polynucleotides are selected from:
(1) a polynucleotide encoding a polypeptide comprising an amino acid sequence having at least 85% (e.g., at least 85%, 90%, 91%, 92%, 93%, 94%, 95%, 96%, 97%, 98%, 99%, or 100%) identity to SEQ ID NO:2 or to SEQ ID NO:3;
(2) a polynucleotide having at least 85% (e.g., at least 85%, 90%, 91%, 92%, 93%, 94%, 95%, 96%, 97%, 98%, 99%, or 100%) identity to SEQ ID NO:1, or hybridizes under medium stringency conditions, high stringency conditions, or very high stringency conditions to SEQ ID NO:1, or to a complementary sequence thereof.

Aspects of the present compositions and methods include methods of making or producing an Ate3C polypeptide having beta-glucosidase activity, employing an isolated nucleic acid sequence encoding the recombinant polypeptide comprising an amino acid sequence that is at least 85% identical (e.g., at least 85%, 90%, 91%, 92%, 93%, 94%, 95%, 96%, 97%, 98%, 99%, or 100%) to that of SEQ ID NO:2, or that of the mature sequence SEQ ID NO:3. In some embodiments, the polypeptide further comprises a native or non-native signal peptide such that the Ate3C polypeptide that is produced is secreted by a host organism, for example, the signal peptide comprises a sequence that is at least 90% identical to SEQ ID NO:13 (the signal sequence of Trichoderma reesei Bgl1). In certain embodiments the isolated nucleic acid comprises a sequence that is at least 85% (e.g., at least 85%, 90%, 91%, 92%, 93%, 94%, 95%, 96%, 97%, 98%, 99%, or 100%) identical to SEQ ID NO:1. In certain embodiments, the isolated nucleic acid further comprises a nucleic acid sequence encoding a signal peptide sequence. In certain embodiments, the signal peptide sequence may be one selected from SEQ ID NOs:13-42. In certain particular embodiments, a nucleic acid sequence encoding the signal peptide sequence of SEQ ID NO:13 is used to express an Ate3C polypeptide in Trichoderma reesei.

Aspects of the present compositions and methods include an expression vector comprising the isolated nucleic acid as described above in operable combination with a regulatory sequence.

Aspects of the present compositions and methods include a host cell comprising the expression vector. In certain embodiments, the host cell is a bacterial cell or a fungal cell. In certain embodiments, the host cell comprising the expression vector is an ethanologen microbe capable of metabolizing the soluble sugars produced from a hydrolysis of a lignocellulosic biomass, wherein the hydrolysis is the result of a chemical and/or enzymatic process.

Aspects of the present compositions and methods include a composition comprising the host cell described above and a culture medium. Aspects of the present compositions and methods include a method of producing an Ate3C polypeptide comprising: culturing the host cell described above in a culture medium, under suitable conditions to produce the beta-glucosidase.

Aspects of the present compositions and methods include a composition comprising an Ate3C polypeptide in the supernatant of a culture medium produced in accordance with the methods for producing the beta-glucosidase as described above.

In some aspects the present invention is related to nucleic acid constructs, recombinant expression vectors, engineered host cells comprising a polynucleotide encoding a polypeptide having beta-glucosidase activity, as described above and herein. In further aspects, the present invention pertains to methods of preparing or producing the beta-glucosidase polypeptides of the invention or compositions comprising such beta-glucosidase polypeptides using the nucleic acid constructs, recombinant expression vectors, and/or engineered host cells. In particular, the present invention is related, for example, to a nucleic acid constructs comprising a suitable signal peptide operably linked to the mature sequence of the beta-glucosidase that is at least 85% identical to SEQ ID NO:2 or to the mature sequence of SEQ ID NO:3, or is encoded by a polynucleotide that is at least 85% identical to SEQ ID NO:1, an isolated polynucleotide, a nucleic acid construct, a recombinant expression vector, or an engineered host cell comprising such a nucleic acid construct. In some embodiments, the signal peptide and beta-glucosidase sequences are derived from different microorganisms.

Also provided is an expression vector comprising the isolated nucleic acid in operable combination with a regulatory sequence. Additionally, a host cell is provided comprising the expression vector. In still further embodiments, a composition is provided, which comprises the host cell and a culture medium.

In some embodiments, the host cell is a bacterial cell or a fungal cell. In certain embodiments, the host cell is an ethanologen microbe, which is capable of metabolizing the soluble sugars produced from hydrolyzing a lignocellulosic biomass substrate, wherein the hydrolyzing can be through a chemical hydrolysis or enzymatic hydrolysis or a combination of these processes, but is also capable of expression of heterologous enzymes. In some embodiments, the host cell is a Saccharomyces cerevisiae or a Zymomonas mobilis cell, which are not only capable of expressing a heterologous polypeptide such as an Ate3C polypeptide of the invention, but also capable of fermenting sugars into ethanol and/or downstream products. In certain particular embodiments, the Saccharomyces cerevisiae cell or Zymomonas mobilis cell, which expresses the beta-glucosidase, is capable of fermenting the sugars produced from a lignocellulosic biomass by an enzyme composition comprising one or more beta-glucosidases. The enzyme composition comprising one or more beta-glucosidases may comprise the same beta-glucosidase or may comprise one or more different beta-glucosidases. In certain embodiments, the enzyme composition comprising one or more beta-glucosidases may be an enzyme mixture produced by an engineered host cell, which may be a bacterial or a fungal cell. When a Saccharomyces cerevisiae or a Zymomonas mobilis cell expressing the Ate3C polypeptide of the present disclosure, the Ate3C polypeptide may be expressed but not secreted. Accordingly the cellobiose must be introduced or “transported” into such a host cell in order for the beta-glucosidase Ate3C polypeptide to catalyze the liberation of D-glucose. Therefore in certain embodiments, the Saccharomyces cerevisiae or a Zymomonas mobilis cell are transformed with a cellobiose transporter gene in addition to one that encodes the Ate3C polypeptide. A cellobiose transporter and a beta-glucosidase have been expressed in Saccharomyces cerevisiae such that the resulting microbe is capable of fermenting cellobiose, for example, in Ha et al., (2011) PNAS, 108(2):504-509. Another cellobiose transporter has been expressed in a Pichia yeast, for example in published U.S. patent application Ser. No. 20110262983. A cellobiose transporter has been introduced into an E. coli, for example, in Sekar et al., (2012) Applied Environmental Microbiology, 78(5):1611-1614.

In further embodiments, the Ate3C polypeptide is heterologously expressed by a host cell. For example, the Ate3C polypeptide is expressed by an engineered microorganism that is not Aspergillus terreus. In some embodiments, the Ate3C polypeptide is co-expressed with one or more different cellulase genes. In some embodiments, the Ate3C polypeptide is co-expressed with one or more hemicellulase genes.

In some aspects, compositions comprising the recombinant Ate3C polypeptides of the preceding paragraphs and methods of preparing such compositions are provided. In some embodiments, the composition further comprises one or more other cellulases, whereby the one or more other cellulases are co-expressed by a host cell with the Ate3C polypeptide. For example, the one or more other cellulases can be selected from no or one or more other beta-glucosidases, one or more cellobiohydrolyases, and/or one or more endoglucanases. Such other beta-glucosidases, cellobiohydrolases and/or endoglucanases, if present, can be co-expressed with the Ate3C polypeptide by a single host cell. At least two of the two or more cellulases may be heterologous to each other or derived from different organisms. For example, the composition may comprise two beta-glucosidases, with the first one being an Ate3C polypeptide, and the second beta-glucosidase being not derived from an Aspergillus terreus strain. For example, the composition may comprise at least one cellobiohydrolase, one endoglucanase, or one beta-glucosidase that is not derived from Aspergillus terreus. In some embodiments, one or more of the cellulases are endogenous to the host cell, but are overexpressed or expressed at a level that is different from that would otherwise be naturally-occurring in the host cell. For example, one or more of the cellulases may be a Trichoderma reesei CBH1 and/or CBH2, which are native to a Trichoderma reesei host cell, but either or both CBH1 and CBH2 are overexpressed or underexpressed when they are co-expressed in the Trichoderma reesei host cell with an Ate3C polypeptide.

In certain embodiments, the composition comprising the recombinant Ate3C polypeptide may further comprise one or more hemicellulases, whereby the one or more hemicellulases are co-expressed by a host cell with the Ate3C polypeptide. For example, the one or more hemicellulases can be selected from one or more xylanase, one or more beta-xylosidases, and/or one or more L-arabinofuranosidases. Such other xylanases, beta-xylosidases and L-arabinofuranosidases, if present, can be co-expressed with the Ate3C polypeptide by a single host cell. In some embodiments, the composition may comprise at least one beta-xylosidase, xylanase or arabinofuranosidase that is not derived from Aspergillus terreus.

In further aspects, the composition comprising the recombinant Ate3C polypeptide may further comprise one or more other cellulases and one or more hemicelluases, whereby the one or more cellulases and/or one or more hemicellulases are co-expressed by a host cell with the Ate3C polypeptide. For example, an Ate3C polypeptide may be co-expressed with one or more other beta-glucosidases, one or more cellobiohydrolases, one or more endoglucanases, one or more endo-xylanases, one or more beta-xylosidases, and one or more L-arabinofuranosidases, in addition to other non-cellulase non-hemicellulase enzymes or proteins in the same host cell. Aspects of the present compositions and methods accordingly include a composition comprising the host cell described above co-expressing a number of enzymes in addition to the Ate3C polypeptide and a culture medium. Aspects of the present compositions and methods accordingly include a method of producing an Ate3C-containing enzyme composition comprising: culturing the host cell, which co-expresses a number of enzymes as described above with the Ate3C polypeptide in a culture medium, under suitable conditions to produce the Ate3C and the other enzymes. Also provided are compositions that comprise the Ate3C polypeptide and the other enzymes produced in accordance with the methods herein in supernatant of the culture medium. Such supernatant of the culture medium can be used as is, with minimum or no post-production processing, which may typically include filtration to remove cell debris, cell-kill procedures, and/or ultrafiltration or other steps to enrich or concentrate the enzymes therein. Such supernatants are called “whole broths” or “whole cellulase broths” herein.

In further aspects, the present invention pertains to a method of applying or using the composition as described above under conditions suitable for degrading or converting a cellulosic material and for producing a substance from a cellulosic material.

In a further aspect, methods for degrading or converting a cellulosic material into fermentable sugars are provided, comprising: contacting the cellulosic material, preferably having already been subject to one or more pretreatment steps, with the Ate3C polypeptides or the compositions comprising such polypeptides of one of the preceding paragraphs to yield fermentable sugars.

Accordingly the instant specification is drawn to the following particular aspects:

In a first aspect, a recombinant polypeptide comprising an amino acid sequence that is at least 85% identical to the amino acid sequence of SEQ ID NO:2 or SEQ ID NO:3, wherein the polypeptide has beta-glucosidase activity.

In a second aspect, the recombinant polypeptide of the first aspect, wherein the polypeptide has improved beta-glucosidase activity as compared to Trichoderma reesei Bgl1 when the recombinant polypeptide and the Trichoderma reesei Bgl1 are used to hydrolyze lignocellulosic biomass substrates.

In a third aspect, the recombinant polypeptide of the first or second aspect, wherein the improved beta-glucosidase activity is an increased cellobiase activity or an improved capacity to hydrolyze cellobiose, thereby liberating D-glucose.

In a fourth aspect, the recombinant polypeptide of any one of the first to third aspects, wherein the improved beta-glucosidase activity is an increased yield of glucose and an equal or lower yield of total sugars from a lignocellulosic biomass under the same saccharification conditions.

In a fifth aspect, the recombinant polypeptide of any one of the first to fourth aspects above, wherein the lignocellulosic biomass is one that has been subject to a pretreatment prior to saccharification. The pretreatment may suitably be those known in the art that renders the lignocellulosic biomass substrate more amenable to the enzymatic access and hydrolysis, which may include, for example those pretreatment methods described herein.

In a sixth aspect, the recombinant polypeptide of any one of the first to fifth aspects above, wherein the polypeptide comprises an amino acid sequence that is at least 90% identical to the amino acid sequence of SEQ ID NO:2 or SEQ ID NO:3.

In a seventh aspect, the recombinant polypeptide of any one of the first to fifth aspects above, wherein the polypeptide comprises an amino acid sequence that is at least 95% identical to the amino acid sequence of SEQ ID NO:2 or SEQ ID NO:3.

In an eighth aspect, a composition comprising the recombinant polypeptide of any one of the first to seventh aspects above, further comprising one or more other cellulases.

In a ninth aspect, the composition of the eighth aspect, wherein the one or more other cellulases are selected from no or one or more other beta-glucosidases, one or more cellobiohydrolases and one or more endoglucanases.

In a tenth aspect, a composition comprising the recombinant polypeptide of any one of the first to seven aspects above, further comprising one or more hemicellulases.

In an eleventh aspect, the composition of the eighth or the ninth aspect as above, further comprising one or more hemicellulases.

In a twelfth aspect, the composition of the tenth or the eleventh aspect as above, wherein the one or more hemicellulases are selected from one or more xylanases, one or more beta-xylosidases, and one or more L-arabinofuranosidases.

In a thirteenth aspect, a nucleic acid encoding the recombinant polypeptide of any one of the first to seventh aspects.

In a fourteenth aspect, the nucleic acid of the thirteenth aspect, further comprising a signal sequence.

In a fifteenth aspect, the nucleic acid of the fourteenth aspect, wherein the signal sequence is selected from the group consisting of SEQ ID NOs: 13-42.

In a sixteenth aspect, an expression vector comprising the nucleic acid of any one of the thirteenth to fifteenth aspects in operable combination with a regulatory sequence.

In a seventeenth aspect, a host cell comprising the expression vector of the sixteenth aspect.

In an eighteenth aspect, the host cell of the seventeenth aspect, wherein the host cell is a bacterial cell or a fungal cell. A number of bacterial cells are known to be suitable host cells as described herein. A number of fungal cells are also suitable. In some embodiments, the bacterial or fungal host cell may be one that is an ethanologen, capable of fermenting or metabolizing certain monomeric sugars into ethanol. For example, the bacterial ethanolgen Zymomonas mobilis may be a host cell expressing a beta-glucosidase polypeptide of the present disclosure. For example, a fungal ethanologen Saccharomyces cerevisiae yeast may also serve as a host cell to produce a beta-glucosidase polypeptide of the present disclosure.

In a nineteenth aspect, a composition comprising the host cell of the sixteenth or the seventeenth aspect and a culture medium.

In a twentieth aspect, a method of producing a beta-glucosidase comprising culturing the host cell of the seventeenth or eighteenth aspect, in a culture medium, under suitable conditions to produce the beta-glucosidase.

In a 21^{st} aspect, a composition comprising the beta-glucosidase produced in accordance with the method of the 20^{th} aspect above, in supernatant of the culture medium.

In a 22^{nd} aspect, a method for hydrolyzing a lignocellulosic biomass substrate, comprising contacting the lignocellulosic biomass substrate with the polypeptide of any one of the first to seventh aspects or with the composition of the 21^{st} aspect, to yield a glucose and/or other sugars.

These and other aspects of Ate3C compositions and methods will be apparent from the following description.

### DESCRIPTION OF THE DRAWINGS

FIG. 1 depicts a map of the pENTR/D-TOPO-Bgl1(943/942) vector.

FIG. 2 depicts a map of the pTrex3g 943/942 construct.

FIGS. 3A-3C provide comparisons hydrolysis performance of Ate3C vs. the benchmark Trichoderma reesei Bgl1 using a phosphoric acid swollen cellulase (PASC) as substrate, at 50° C., and 1.5 h, wherein the Ate3C and Bgl1 were added to a background whole cellulase produced from an engineered Trichoderma reesei strain in accordance with what is described in Published Patent Application WO 2011/038019, and the beta-glucosidase+whole cellulase mixture was mixed with the PASC substrate at various beta-glucosidase doses. FIG. 3A depicts the measurements and comparison of glucan conversion at various beta-glucosidase doses. FIG. 3B depicts the measurements and comparison of total glucose production at various beta-glucosidase doses. FIG. 3C depicts the measurements and comparison of cellobiose produced from hydrolyzing the PASC at various beta-glucosidase doses.

FIGS. 4A-4B provide comparison of hydrolysis performance of Ate3C vs. the benchmark Trichoderma reesei Bgl1 using a dilute ammonia pretreated corn stover (DACS) as substrate, at 50° C. for 2 days, wherein the Ate3C and Bgl1 were added to a background whole cellulase produced from an engineered Trichoderma reesei strain in accordance with what is described in Published Patent Application WO 2011/038019, and the beta-glucosidase+cellulase mixture was mixed with the DACS substrate at various total protein to cellulose doses. FIG. 4A depicts the measurements and comparison of total glucan conversion at various total protein to cellulose doses. FIG. 4B depicts the measurements and comparison of total glucose production at various total protein to cellulose doses.

FIGS. 5A-5B provide comparison of hydrolysis performance of Ate3C vs. the benchmark Trichoderma reesei Bgl1 using a dilute ammonia pretreated corn stover (DACS) as substrate, at 50° C. for 2 days, wherein various doses of Ate3C and Bgl1 were added to a whole cellulase that is used at a constant 13.4 mg protein/g cellulose, produced from an engineered Trichoderma reesei strain in accordance with what is described in Published Patent Application WO 2011/038019. FIG. 5A depicts the measurements and comparison of total glucan conversion at various beta-glucosidase loading (with the whole cellulase background being held at the constant 13.4 mg protein/g cellulose). FIG. 5B depicts the measurements and comparison of total glucose production at various beta-glucosidase loading (with the whole cellulase background being held at the constant 13.4 mg protein/g cellulose)

FIG. 6 depicts a yeast shuttle vector pSC11 construct comprising an Ate3C gene optimized and synthesized for expression of the Ate3C polypeptide in a Saccharomyces cerevisiae ethanologen.

FIG. 7 depicts a Zymomonas mobilis integration vector pZC11 comprising an Ate3C gene optimized and synthesized for expression of the Ate3C polypeptide in a Zymomonas mobilis ethanologen.

FIGS. 8A-8D depict the sequences and sequence identifiers of the present disclosure.

### DETAILED DESCRIPTION

### I. Overview

Described herein are compositions and methods relating to a recombinant beta-glucosidase Ate3C belonging to glycosyl hydrolase family 3 from Aspergillus terreus. The present compositions and methods are based, in part, on the observations that recombinant Ate3C polypeptides have higher cellulase activities and are more robust as a component of an enzyme composition when the composition is used to hydrolyze a lignocellulosic biomass material or feedstock than, for example, a known benchmark high fidelity beta-glucosidase Bgl1 of Trichoderma reesei. These features of Ate3C polypeptides make them, or variants thereof, suitable for use in numerous processes, including, for example, in the conversion or hydrolysis of a lignocellulosic biomass feedstock.

Before the present compositions and methods are described in greater detail, it is to be understood that the present compositions and methods are not limited to particular embodiments described, as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior invention. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “recombinant,” when used in reference to a subject cell, nucleic acid, polypeptides/enzymes or vector, indicates that the subject has been modified from its native state. Thus, for example, recombinant cells express genes that are not found within the native (non-recombinant) form of the cell, or express native genes at different levels or under different conditions than found in nature. Recombinant nucleic acids may differ from a native sequence by one or more nucleotides and/or are operably linked to heterologous sequences, e.g., a heterologous promoter, signal sequences that allow secretion, etc., in an expression vector. Recombinant polypeptides/enzymes may differ from a native sequence by one or more amino acids and/or are fused with heterologous sequences. A vector comprising a nucleic acid encoding a beta-glucosidase is, for example, a recombinant vector.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of”are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

### II. Definitions

“Beta-glucosidase” refers to a beta-D-glucoside glucohydrolase of E.C. 3.2.1.21. The term “beta-glucosidase activity” therefore refers the capacity of catalyzing the hydrolysis of beta-D-glucose or cellobiose to release D-glucose. Beta-glucosidase activity may be determined using a cellobiase assay, for example, which measures the capacity of the enzyme to catalyze the hydrolysis of a cellobiose substrate to yield D-glucose, as described in Example 2C of the present disclosure.

As used herein, “Ate3C” or “an Ate3C polypeptide” refers to a beta-glucosidase belonging to glycosyl hydrolase family 3 (e.g., a recombinant beta-glucosidase) derived from Aspergillus terreus (and variants thereof), that has improved performance hydrolyzing a lignocellulosic biomass substrate when compared to a benchmark beta-glucosidase, the wild type Trichoderma reesei Bgl1 polypeptide having the amino acid sequence of SEQ ID NO:4. According to aspects of the present compositions and methods, Ate3C polypeptides include those having the amino acid sequence depicted in SEQ. ID NO:2, as well as derivative or variant polypeptides having at least 85%, at least 90%, at least 91%, at least 92%, at least 93%, at least 94%, at least 95%, at least 96%, at least 97%, at least 98%, or at least 99% sequence identity to the amino acid sequence of SEQ. ID NO:2, or to the mature sequence SEQ ID NO:2, or to a fragment of at least 100 residues in length of SEQ. ID NO:2, wherein the Ate3C polypeptides not only have beta-glucosidase activity and capable of catalyzing the conversion of cellobiose into D-glucose, but also have higher beta-glucosidase activity and have higher capacity to catalyze the conversion of cellobiose to D-glucose than Trichoderma reesei Bgl1.

The Ate3C polypeptides to be used in the compositions and methods of the present disclosure would have at least 10%, preferably at least 20%, more preferably at least 30%, and even more preferably at least 40%, more preferably at least 50%, even more preferably at least 60%, and preferably at least 70%, more preferably at least 90%, even more preferably at least 100% or more of the beta-glucosidase activity of the polypeptide of the amino acid sequence of SEQ ID NO:2, or of the polypeptide consisting of residues 20 to 861 of the SEQ ID NO:2; or of the mature sequence SEQ ID NO:3.

“Family 3 glycosyl hydrolase” or “GH3” refers to polypeptides falling within the definition of glycosyl hydrolase family 3 according to the classification by Henrissat, Biochem. J. 280:309-316 (1991), and by Henrissat & Cairoch, Biochem. J., 316:695-696 (1996).

Ate3C polypeptides according to the present compositions and methods described herein can be isolated or purified. By purification or isolation is meant that the Ate3C polypeptide is altered from its natural state by virtue of separating the Ate3C from some or all of the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to the Ate3C-containing composition which provide additional benefits, for example, activating agents, anti-inhibition agents, desirable ions, compounds to control pH or other enzymes or chemicals.

As used herein, “microorganism” refers to a bacterium, a fungus, a virus, a protozoan, and other microbes or microscopic organisms.

As used herein, a “derivative” or “variant” of a polypeptide means a polypeptide, which is derived from a precursor polypeptide (e.g., the native polypeptide) by addition of one or more amino acids to either or both the C- and N-terminal end, substitution of one or more amino acids at one or a number of different sites in the amino acid sequence, deletion of one or more amino acids at either or both ends of the polypeptide or at one or more sites in the amino acid sequence, or insertion of one or more amino acids at one or more sites in the amino acid sequence. The preparation of an Ate3C derivative or variant may be achieved in any convenient manner, e.g., by modifying a DNA sequence which encodes the native polypeptides, transformation of that DNA sequence into a suitable host, and expression of the modified DNA sequence to form the derivative/variant Ate3C. Derivatives or variants further include Ate3C polypeptides that are chemically modified, e.g., glycosylation or otherwise changing a characteristic of the Ate3C polypeptide. While derivatives and variants of Ate3C are encompassed by the present compositions and methods, such derivates and variants will display improved beta-glucosidase activity when compared to that of the wild type Trichoderma reesei Bgl1 of SEQ ID NO:4, under the same lignocellulosic biomass substrate hydrolysis conditions. In certain aspects, an Ate3C polypeptide of the compositions and methods herein may also encompasses functional fragment of a polypeptide or a polypeptide fragment having beta-glucosidase activity, which is derived from a parent polypeptide, which may be the full length polypeptide comprising or consisting of SEQ ID NO:2, or the mature sequence comprising or consisting SEQ ID NO:3. The functional polypeptide may have been truncated either in the N-terminal region, or the C-terminal region, or in both regions to generate a fragment of the parent polypeptide. For the purpose of the present disclosure, a functional fragment must have at least 20%, more preferably at least 30%, 40%, 50%, or preferably, at least 60%, 70%, 80%, or even more preferably at least 90% of the beta-glucosidase activity of that of the parent polypeptide.

In certain aspects, an Ate3C derivative/variant will have anywhere from 85% to 99% (or more) amino acid sequence identity to the amino acid sequence of SEQ. ID NO:2, or to the mature sequence SEQ ID NO:3, e.g., 85%, 86%, 87%, 88%, 89%, 90%, 91%, 92%, 93%, 94%, 95%, 96%, 97%, 98%, or 99% amino acid sequence identity to the amino acid sequence of SEQ. ID NO:2 or to the mature sequence SEQ ID NO:3. In some embodiments, amino acid substitutions are “conservative amino acid substitutions” using L-amino acids, wherein one amino acid is replaced by another biologically similar amino acid. Conservative amino acid substitutions are those that preserve the general charge, hydrophobicity/hydrophilicity, and/or steric bulk of the amino acid being substituted. Examples of conservative substitutions are those between the following groups: Gly/Ala, Val/Ile/Leu, Lys/Arg, Asn/Gln, Glu/Asp, Ser/Cys/Thr, and Phe/Trp/Tyr. A derivative may, for example, differ by as few as 1 to 10 amino acid residues, such as 6-10, as few as 5, as few as 4, 3, 2, or even 1 amino acid residue. In some embodiments, an Ate3C derivative may have an N-terminal and/or C-terminal deletion, where the Ate3C derivative excluding the deleted terminal portion(s) is identical to a contiguous sub-region in SEQ ID NO: 2 or SEQ ID NO:3.

As used herein, “percent (%) sequence identity” with respect to the amino acid or nucleotide sequences identified herein is defined as the percentage of amino acid residues or nucleotides in a candidate sequence that are identical with the amino acid residues or nucleotides in an Ate3C sequence, after aligning the sequences and introducing gaps, if necessary, to achieve the maximum percent sequence identity, and not considering any conservative substitutions as part of the sequence identity.

By “homologue” shall mean an entity having a specified degree of identity with the subject amino acid sequences and the subject nucleotide sequences. A homologous sequence is taken to include an amino acid sequence that is at least 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologues will include the same active site residues as the subject amino acid sequence, unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J. Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J. Mol. Biol. 215:403-410).

Computerized programs using these algorithms are also available, and include, but are not limited to: ALIGN or Megalign (DNASTAR) software, or WU-BLAST-2 (Altschul et al., (1996) Meth. Enzym., 266:460-480); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared. Preferably, the sequence identity is determined using the default parameters determined by the program. Specifically, sequence identity can determined by using Clustal W (Thompson J. D. et al. (1994) Nucleic Acids Res. 22:4673-4680) with default parameters, i.e.:

Gap opening penalty: 10.0

Gap extension penalty: 0.05

Protein weight matrix: BLOSUM series

DNA weight matrix: IUB

Delay divergent sequences %: 40

Gap separation distance: 8

DNA transitions weight: 0.50

List hydrophilic residues: GPSNDQEKR

Use negative matrix: OFF

Toggle Residue specific penalties: ON

Toggle hydrophilic penalties: ON

Toggle end gap separation penalty OFF

As used herein, “expression vector” means a DNA construct including a DNA sequence which is operably linked to a suitable control sequence capable of affecting the expression of the DNA in a suitable host. Such control sequences may include a promoter to affect transcription, an optional operator sequence to control transcription, a sequence encoding suitable ribosome-binding sites on the mRNA, and sequences which control termination of transcription and translation. Different cell types may be used with different expression vectors. An exemplary promoter for vectors used in Bacillus subtilis is the AprE promoter; an exemplary promoter used in Streptomyces lividans is the A4 promoter (from Aspergillus niger); an exemplary promoter used in E. coli is the Lac promoter, an exemplary promoter used in Saccharomyces cerevisiae is PGK1, an exemplary promoter used in Aspergillus niger is glaA, and an exemplary promoter for Trichoderma reesei is cbhl. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the present compositions and methods are intended to include other forms of expression vectors which serve equivalent functions and which are, or become, known in the art. Thus, a wide variety of host/expression vector combinations may be employed in expressing the DNA sequences described herein. Useful expression vectors, for example, may consist of segments of chromosomal, non-chromosomal and synthetic DNA sequences such as various known derivatives of SV40 and known bacterial plasmids, e.g., plasmids from E. coli including col E1, pCR1, pBR322, pMb9, pUC 19 and their derivatives, wider host range plasmids, e.g., RP4, phage DNAs e.g., the numerous derivatives of phage λ, e.g., NM989, and other DNA phages, e.g., M13 and filamentous single stranded DNA phages, yeast plasmids such as the 2 μ plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present compositions and methods are known in the art and are described generally in, for example, Sambrook et al., Molecular Cloning: A Laboratory Manual, Second Edition, Cold Spring Harbor Press (1989). Often, such expression vectors including the DNA sequences described herein are transformed into a unicellular host by direct insertion into the genome of a particular species through an integration event (see e.g., Bennett & Lasure, More Gene Manipulations in Fungi, Academic Press, San Diego, pp. 70-76 (1991) and articles cited therein describing targeted genomic insertion in fungal hosts).

As used herein, “host strain” or “host cell” means a suitable host for an expression vector including DNA according to the present compositions and methods. Host cells useful in the present compositions and methods are generally prokaryotic or eukaryotic hosts, including any transformable microorganism in which expression can be achieved. Specifically, host strains may be Bacillus subtilis, Streptomyces lividans, Escherichia coli, Trichoderma reesei, Saccharomyces cerevisiae or Aspergillus niger. In certain embodiments, the host cell may be an ethanologen microbe, which may be, for example, a yeast such as Saccharomyces cerevisiae or a bacterium ethanologen such as a Zymomonas mobilis. When a Saccharomyces cerevisiae or Zymomonas mobilis is used as the host c1ell, and if the beta-glucosidase gene is not made to secret from host cell but is expressed intracellularly, a cellobiose transporter gene can be introduced into the host cell in order to allow the intracellularly expressed beta-glucosidase to act upon the cellobiose substrate and liberate glucose, which will then be metabolized subsequently or immediately by the microorganisms and converted into ethanol.

Host cells are transformed or transfected with vectors constructed using recombinant DNA techniques. Such transformed host cells may be capable of one or both of replicating the vectors encoding Ate3C (and its derivatives or variants (mutants)) and expressing the desired peptide product. In certain embodiments according to the present compositions and methods, “host cell” means both the cells and protoplasts created from the cells of Trichoderma sp.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a beta-glucosidase) has been introduced. Exemplary host strains are microbial cells (e.g., bacteria, filamentous fungi, and yeast) capable of expressing the polypeptide of interest. The term “host cell” includes protoplasts created from cells.

The term “heterologous” with reference to a polynucleotide or polypeptide refers to a polynucleotide or polypeptide that does not naturally occur in a host cell.

The term “endogenous” with reference to a polynucleotide or polypeptide refers to a polynucleotide or polypeptide that occurs naturally in the host cell.

The term “expression” refers to the process by which a polypeptide is produced based on a nucleic acid sequence. The process includes both transcription and translation.

Accordingly the process of converting a lignocellulosic biomass substrate to an ethanol can, in some embodiments, comprise two beta-glucosidase activities. For example, a first beta-glucosidase activity may be applied to the lignocellulosic biomass substrate during the saccharification or hydrolysis step, and a second beta-glucosidase activity can be applied as part of the ethanologen microbe in the fermentation step during which the monomeric or fermentable sugars that resulted from the saccharification or hydrolysis step are metaloblized. The first and second beta-glucosidase activities may, in some embodiments, result from the presence of the same beta-glucosidase polypeptide. For example, the first beta-glucosidase activity in the saccharification may result from the presence of an Ate3C polypeptide of the invention, whereas the second beta-glucosidase activity in the fermentation stage may result from the expression of a different beta-glucosidase by the ethanologen microbe. In another example, the first and second beta-glucosidase activities may result from the presence of the same polypeptide in the saccharification or hydrolysis step and the fermentation step. For example, the same Ate3C polypeptide of the invention may, in some embodiments, provide the beta-glucosidase activities for both the hydrolysis or saccharification step and the fermentation step.

In certain other embodiments, the process of converting a lignocellulosic biomass substrate to an ethanol can, comprise two beta-glucosidase activities whereas the saccharification or hydrolysis step and the fermentation step occurs simultaneously, for example, in the same tank. Two or more beta-glucosidase polypeptides may contribute to the beta-glucosidase activities, one of which may be an Ate3C polypeptide of the invention.

In certain further embodiments, the process of converting a lignocellulosic biomass to an ethanol can comprise a single beta-glucosidase activity whereas either the saccharification or hydrolysis step or the fermentation step, but not both steps involves the participation of a beta-glucosidase. For example, an Ate3C polypeptide of the invention or a composition comprising the Ate3C polypeptide may be used in the saccharification step. In another example, the enzyme composition that is used to hydrolyze the lignocellulosic biomass substrate does not comprise a beta-glucosidase activity, whereas the ethanologen microbe expresses a beta-glucosidase polypeptide, for example, an Ate3C polypeptide of the invention.

As used herein, “signal sequence” means a sequence of amino acids bound to the N-terminal portion of a polypeptide which facilitates the secretion of the mature form of the polypeptide outside of the cell. This definition of a signal sequence is a functional one. The mature form of the extracellular polypeptide lacks the signal sequence which is cleaved off during the secretion process. While the native signal sequence of Ate3C may be employed in aspects of the present compositions and methods, other non-native signal sequences may be employed (e.g., SEQ ID NO: 13). The term “mature,” when referring to a polypeptide herein, is meant a polypeptide in its final form(s) following translation and any post-translational modifications. For example, the Ate3C polypeptides of the invention has one or more mature forms, at least one of which has the amino acid sequence of SEQ ID NO:3.

The beta-glucosidase polypeptides of the invention may be referred to as “precursor,” “immature,” or “full-length,” in which case they include a signal sequence, or may be referred to as “mature,” in which case they lack a signal sequence. Mature forms of the polypeptides are generally the most useful. Unless otherwise noted, the amino acid residue numbering used herein refers to the mature forms of the respective amylase polypeptides. The beta-glucosidase polypeptides of the invention may also be truncated to remove the N or C-termini, so long as the resulting polypeptides retain beta-glucosidase activity.

The beta-glucosidase polypeptides of the invention may also be a “chimeric” or “hybrid” polypeptide, in that it includes at least a portion of a first beta-glucosidase polypeptide, and at least a portion of a second beta-glucosidase polypeptide (such chimeric beta-glucosidase polypeptides may, for example, be derived from the first and second beta-glucosidases using known technologies involving the swapping of domains on each of the beta-glucosidases). The present beta-glucosidase polypeptides may further include heterologous signal sequence, an epitope to allow tracking or purification, or the like. When the term of “heterologous” is used to refer to a signal sequence used to express a polypeptide of interest, it is meant that the signal sequence is, for example, derived from a different microorganism as the polypeptide of interest. Examples of suitable heterologous signal sequences for expressing the Ate3C polypeptides herein, may be, for example, those from Trichoderma reesei.

As used herein, “functionally attached” or “operably linked” means that a regulatory region or functional domain having a known or desired activity, such as a promoter, terminator, signal sequence or enhancer region, is attached to or linked to a target (e.g., a gene or polypeptide) in such a manner as to allow the regulatory region or functional domain to control the expression, secretion or function of that target according to its known or desired activity.

As used herein, the terms “polypeptide” and “enzyme” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, “wild-type” and “native” genes, enzymes, or strains, are those found in nature.

The terms “wild-type,” “parental,” or “reference,” with respect to a polypeptide, refer to a naturally-occurring polypeptide that does not include a man-made substitution, insertion, or deletion at one or more amino acid positions. Similarly, the term “wild-type,” “parental,” or “reference,” with respect to a polynucleotide, refers to a naturally-occurring polynucleotide that does not include a man-made nucleoside change. However, a polynucleotide encoding a wild-type, parental, or reference polypeptide is not limited to a naturally-occurring polynucleotide, but rather encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues. They may be defined by their level of primary amino acid sequence homology/identity with a parent polypeptide. Suitably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity to a parent polypeptide.

As used herein, a “variant polynucleotide” encodes a variant polypeptide, has a specified degree of homology/identity with a parent polynucleotide, or hybridized under stringent conditions to a parent polynucleotide or the complement thereof. Suitably, a variant polynucleotide has at least 80%, at least 85%, at least 90%, at least 91%, at least 92%, at least 93%, at least 94%, at least 95%, at least 96%, at least 97%, at least 98%, or even at least 99% nucleotide sequence identity to a parent polynucleotide or to a complement of the parent polynucleotide. Methods for determining percent identity are known in the art and described above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm −5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Alternatively, or in addition, hybridization conditions can be based upon the salt or ionic strength conditions of hybridization, and/or upon one or more stringency washes, e.g., 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used).

As used herein, the term “hybridization” refers to the process by which a strand of nucleic acid joins with a complementary strand through base pairing, as known in the art. More specifically, “hybridization” refers to the process by which one strand of nucleic acid forms a duplex with, i.e., base pairs with, a complementary strand, as occurs during blot hybridization techniques and PCR techniques. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while intermediate or low stringency hybridization can be used to identify or detect polynucleotide sequence homologs.

Intermediate and high stringency hybridization conditions are well known in the art. For example, intermediate stringency hybridizations may be carried out with an overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate and 20 mg/ml denatured sheared salmon sperm DNA, followed by washing the filters in 1×SSC at about 37-50° C. High stringency hybridization conditions may be hybridization at 65° C. and 0.1×SSC (where 1×SSC=0.15 M NaCl, 0.015 M Na₃ citrate, pH 7.0). Alternatively, high stringency hybridization conditions can be carried out at about 42° C. in 50% formamide, 5×SSC, 5× Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. And very high stringent hybridization conditions may be hybridization at 68° C. and 0.1×SSC. Those of skill in the art know how to adjust the temperature, ionic strength, etc. as necessary to accommodate factors such as probe length and the like.

A nucleic acid encoding a variant beta-glucosidase may have a Tₘ reduced by 1° C.-3° C. or more compared to a duplex formed between the nucleotide of SEQ ID NO: 1 and its identical complement.

The phrase “substantially similar” or “substantially identical,” in the context of at least two nucleic acids or polypeptides, means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences may include a promoter to effect transcription, an optional operator sequence to control such transcription, a sequence encoding suitable mRNA ribosome binding sites and/or sequences that control termination of transcription and translation. The vector may be a plasmid, a phage particle, or a potential genomic insert. Once transformed into a suitable host, the vector may replicate and function independently of the host genome, or may, in some instances, integrate into the host genome.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in a manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the polypeptide from the cell. The mature form of the extracellular polypeptide lacks the signal sequence which is cleaved off during the secretion process.

The term “selective marker” or “selectable marker,” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element,” refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally cells of prokaryotic or eukaryotic hosts that are transformed or transfected with vectors constructed using recombinant DNA techniques known in the art. Transformed host cells are capable of either replicating vectors encoding the polypeptide variants or expressing the desired polypeptide variant. In the case of vectors, which encode the pre- or pro-form of the polypeptide variant, such variants, when expressed, are typically secreted from the host cell into the host cell medium.

The term “introduced,” in the context of inserting a nucleic acid sequence into a cell, means transformation, transduction, or transfection. Means of transformation include protoplast transformation, calcium chloride precipitation, electroporation, naked DNA, and the like as known in the art. (See, Chang and Cohen (1979) Mol. Gen. Genet. 168:111-115; Smith et al., (1986) Appl. Env. Microbiol. 51:634; and the review article by Ferrari et al., in Harwood, Bacillus, Plenum Publishing Corporation, pp. 57-72, 1989).

“Fused” polypeptide sequences are connected, i.e., operably linked, via a peptide bond between two subject polypeptide sequences.

The term “filamentous fungi” refers to all filamentous forms of the subdivision Eumycotina, particulary Pezizomycotina species.

An “ethanologenic microorganism” refers to a microorganism with the ability to convert a sugar or oligosaccharide to ethanol.

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991).

### III. Beta-glucosidase Polypeptides, Polynucleotides, Vectors, and Host Cells

A. Ate3C Polypeptides

In one aspect, the present compositions and methods provide a recombinant Ate3C beta-glucosidase polypeptide, fragments thereof, or variants thereof having beta-glucosidase activity. An example of a recombinant beta-glucosidase polypeptide was isolated from Aspergillus terreus. The mature Ate3C polypeptide has the amino acid sequence set forth as SEQ ID NO:3. Similar, substantially similar Ate3C polypeptides may occur in nature, e.g., in other strains or isolates of Aspergillus terreus. These and other recombinant Ate3C polypeptides are encompassed by the present compositions and methods.

In some embodiments, the recombinant Ate3C polypeptide is a variant Ate3C polypeptide having a specified degree of amino acid sequence identity to the exemplified Ate3C polypeptide, e.g., at least 85%, 90%, 91%, 92%, 93%, 94%, 95%, 96%, 97%, 98%, or even at least 99% sequence identity to the amino acid sequence of SEQ ID NO:2 or to the mature sequence SEQ ID NO:3. Sequence identity can be determined by amino acid sequence alignment, e.g., using a program such as BLAST, ALIGN, or CLUSTAL, as described herein.

In certain embodiments, the recombinant Ate3C polypeptides are produced recombinantly, in a microorganism, for example, in a bacterial or fungal host organism, while in others the Ate3C polypeptides are produced synthetically, or are purified from a native source (e.g., Aspergillus terreus ).

In certain embodiments, the recombinant Ate3C polypeptide includes substitutions that do not substantially affect the structure and/or function of the polypeptide. Examples of these substitutions are conservative mutations, as summarized in Table I.

TABLE I
Amino Acid Substitutions

| Original Residue | Code | Acceptable Substitutions |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, D-Met, |
| | | D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D-Met, Ile, |
| | | D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4, |
| | | or 5-phenylproline, cis-3,4, |
| | | or 5-phenylproline |
| Proline | P | D-Pro, L-I-thioazolidine-4-carboxylic acid, D-or L-1- |
| | | oxazolidine-4-carboxylic acid |
| Serine | S | D-Ser, Thr, D-Thr, allo-Thr, Met, D-Met, Met(O), D-Met(O), |
| | | L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant Ate3C polypeptide, and then expressing the variant polypeptide in an organism. Substitutions involving non-naturally occurring amino acids or chemical modifications to amino acids are generally made by chemically modifying an Ate3C polypeptide after it has been synthesized by an organism.

In some embodiments, variant recombinant Ate3C polypeptides are substantially identical to SEQ ID NO:2 or SEQ ID NO:3, meaning that they do not include amino acid substitutions, insertions, or deletions that do not significantly affect the structure, function, or expression of the polypeptide. Such variant recombinant Ate3C polypeptides will include those designed to circumvent the present description. In some embodiments, variants recombinant Ate3C polypeptides, compositions and methods comprising these variants are not substantially identical to SEQ ID NO:2 or SEQ ID NO:3, but rather include amino acid substitutions, insertions, or deletions that affect, in certain circumstances, substantially, the structure, function, or expression of the polypeptide herein such that improved characteristics, including, e.g., improved specific activity to hydrolyze a lignocellulosic substrate, improved expression in a desirable host organism, improved thermostability, pH stability, etc, as compared to that of a polypeptide of SEQ ID NO:2 or SEQ ID NO:3 can be achieved.

In some embodiments, the recombinant Ate3C polypeptide (including a variant thereof) has beta-glucosidase activity. Beta-glucosidase activity can be determined and measured using the assays described herein, for example, those described in Example 2, or by other assays known in the art.

Recombinant Ate3C polypeptides include fragments of “full-length” Ate3C polypeptides that retain beta-glucosidase activity. Preferably those functional fragments (i.e., fragments that retain beta-glucosidase activity) are at least 100 amino acid residues in length (e.g., at least 100 amino acid residues, at least 120 amino acid residues, at least 140 amino acid residues, at least 160 amino acid residues, at least 180 amino acid residues, at least 200 amino acid residues, at least 220 amino acid residues, at least 240 amino acid residues, at least 260 amino acid residues, at least 280 amino acid residues, at least 300 amino acid residues, at least 320 amino acid residues, or at least 350 amino acid residues in length or longer). Such fragments suitably retain the active site of the full-length precursor polypeptides or full length mature polypeptides but may have deletions of non-critical amino acid residues. The activity of fragments can be readily determined using the assays described herein, for example those described in Example 2, or by other assays known in the art.

In some embodiments, the Ate3C amino acid sequences and derivatives are produced as an N- and/or C-terminal fusion protein, for example, to aid in extraction, detection and/or purification and/or to add functional properties to the Ate3C polypeptides. Examples of fusion protein partners include, but are not limited to, glutathione-S-transferase (GST), 6× His, GAL4 (DNA binding and/or transcriptional activation domains), FLAG-, MYC-tags or other tags known to those skilled in the art. In some embodiments, a proteolytic cleavage site is provided between the fusion protein partner and the polypeptide sequence of interest to allow removal of fusion sequences. Suitably, the fusion protein does not hinder the activity of the recombinant Ate3C polypeptide. In some embodiments, the recombinant Ate3C polypeptide is fused to a functional domain including a leader peptide, propeptide, binding domain and/or catalytic domain. Fusion proteins are optionally linked to the recombinant Ate3C polypeptide through a linker sequence that joins the Ate3C polypeptide and the fusion domain without significantly affecting the properties of either component. The linker optionally contributes functionally to the intended application.

The present disclosure provides host cells that are engineered to express one or more Ate3C polypeptides of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus,Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Methods of transforming nucleic acids into these organisms are known in the art. For example, a suitable procedure for transforming Aspergillus host cells is described in EP 238 023.

In some embodiments, the recombinant Ate3C polypeptide is fused to a signal peptide to, for example, facilitate extracellular secretion of the recombinant Ate3C polypeptide. For example, in certain embodiments, the signal peptide is encoded by a sequence selected from SEQ ID NOs:13-42. In particular embodiments, the recombinant Ate3C polypeptide is expressed in a heterologous organism as a secreted polypeptide. The compositions and methods herein thus encompass methods for expressing an Ate3C polypeptide as a secreted polypeptide in a heterologous organism. In some embodiments the recombinant Ate3C polypeptide is expressed in a heterologous organism intracellularly, for example, when the heterologous organism is an ethanologen microbe such as a Saccharomyces cerevisiaeor a Zymomonas mobilis. In those cases, a cellibiose transporter gene can be introduced into the organism using genetic engineering tools, in order for the Ate3C polypeptide to act on the cellobiose substrate inside the organism to convert cellobiose into D-glucose, which is then metabolized or converted by the organism into ethanol.

The disclosure also provides expression cassettes and/or vectors comprising the above-described nucleic acids. Suitably, the nucleic acid encoding an Ate3C polypeptide of the disclosure is operably linked to a promoter. Promoters are well known in the art. Any promoter that functions in the host cell can be used for expression of a beta-glucosidase and/or any of the other nucleic acids of the present disclosure. Initiation control regions or promoters, which are useful to drive expression of a beta-glucosidase nucleic acids and/or any of the other nucleic acids of the present disclosure in various host cells are numerous and familiar to those skilled in the art (see, for example, WO 2004/033646 and references cited therein). Virtually any promoter capable of driving these nucleic acids can be used.

Specifically, where recombinant expression in a filamentous fungal host is desired, the promoter can be a filamentous fungal promoter. The nucleic acids can be, for example, under the control of heterologous promoters. The nucleic acids can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or beta-glucosidase promoter. A particularly suitable promoter can be, for example, a T. reesei cellobiohydrolase, endoglucanase, or beta-glucosidase promoter. For example, the promoter is a cellobiohydrolase I (cbh1) promoter. Non-limiting examples of promoters include a cbh1, cbh2, egl1, egl2, egl3, egl4, egl5, pki1, gpd1, xyn1, or xyn2 promoter. Additional non-limiting examples of promoters include a T. reesei cbh1, cbh2, egl1, egl2, egl3, egl4, egl5, pki1, gpd1, xyn1, or xyn2 promoter.

The nucleic acid sequence encoding an Ate3C polypeptide herein can be included in a vector. In some aspects, the vector contains the nucleic acid sequence encoding the Ate3C polypeptide under the control of an expression control sequence. In some aspects, the expression control sequence is a native expression control sequence. In some aspects, the expression control sequence is a non-native expression control sequence. In some aspects, the vector contains a selective marker or selectable marker. In some aspects, the nucleic acid sequence encoding the Ate3C polypeptide is integrated into a chromosome of a host cell without a selectable marker.

Suitable vectors are those which are compatible with the host cell employed. Suitable vectors can be derived, for example, from a bacterium, a virus (such as bacteriophage T7 or a M-13 derived phage), a cosmid, a yeast, or a plant. Suitable vectors can be maintained in low, medium, or high copy number in the host cell. Protocols for obtaining and using such vectors are known to those in the art (see, for example, Sambrook et al., Molecular Cloning: A Laboratory Manual, 2^{nd} ed., Cold Spring Harbor, 1989).

In some aspects, the expression vector also includes a termination sequence. Termination control regions may also be derived from various genes native to the host cell. In some aspects, the termination sequence and the promoter sequence are derived from the same source.

A nucleic acid sequence encoding an Ate3C polypeptide can be incorporated into a vector, such as an expression vector, using standard techniques (Sambrook et al., Molecular Cloning: A Laboratory Manual, Cold Spring Harbor, 1982).

In some aspects, it may be desirable to over-express an Ate3C polypeptide and/or one or more of any other nucleic acid described in the present disclosure at levels far higher than currently found in naturally-occurring cells. In some embodiments, it may be desirable to under-express (e.g., mutate, inactivate, or delete) an endogenous beta-glucosidase and/or one or more of any other nucleic acid described in the present disclosure at levels far below that those currently found in naturally-occurring cells.

B. Ate3c Polynucleotides

Another aspect of the compositions and methods described herein is a polynucleotide or a nucleic acid sequence that encodes a recombinant Ate3C polypeptide (including variants and fragments thereof) having beta-glucosidase activity. In some embodiments the polynucleotide is provided in the context of an expression vector for directing the expression of an Ate3C polypeptide in a heterologous organism, such as one identified herein. The polynucleotide that encodes a recombinant Ate3C polypeptide may be operably-linked to regulatory elements (e.g., a promoter, terminator, enhancer, and the like) to assist in expressing the encoded polypeptides.

An example of a polynucleotide sequence encoding a recombinant Ate3C polypeptide has the nucleotide sequence of SEQ ID NO: 1. Similar, including substantially identical, polynucleotides encoding recombinant Ate3C polypeptides and variants may occur in nature, e.g., in other strains or isolates of Aspergillus terreus, or Aspergillus sp. In view of the degeneracy of the genetic code, it will be appreciated that polynucleotides having different nucleotide sequences may encode the same Ate3C polypeptides, variants, or fragments.

In some embodiments, polynucleotides encoding recombinant Ate3C polypeptides have a specified degree of amino acid sequence identity to the exemplified polynucleotide encoding an Ate3C polypeptide, e.g., at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% sequence identity to the amino acid sequence of SEQ ID NO: 2. Homology can be determined by amino acid sequence alignment, e.g., using a program such as BLAST, ALIGN, or CLUSTAL, as described herein.

In some embodiments, the polynucleotide that encodes a recombinant Ate3C polypeptide is fused in frame behind (i.e., downstream of) a coding sequence for a signal peptide for directing the extracellular secretion of a recombinant Ate3C polypeptide. As described herein, the term “heterologous” when used to refer to a signal sequence used to express a polypeptide of interest, it is meant that the signal sequence and the polypeptide of interest are from different organisms. Heterologous signal sequences include, for example, those from other fungal cellulase genes, such as, e.g., the signal sequence of Trichoderma reesei Bgl1, of SEQ ID NO:13. Expression vectors may be provided in a heterologous host cell suitable for expressing a recombinant Ate3C polypeptide, or suitable for propagating the expression vector prior to introducing it into a suitable host cell.

In some embodiments, polynucleotides encoding recombinant Ate3C polypeptides hybridize to the polynucleotide of SEQ ID NO: 1 (or to the complement thereof) under specified hybridization conditions. Examples of conditions are intermediate stringency, high stringency and extremely high stringency conditions, which are described herein.

Ate3C polynucleotides may be naturally occurring or synthetic (i.e., man-made), and may be codon-optimized for expression in a different host, mutated to introduce cloning sites, or otherwise altered to add functionality.

C. Ate3C Vectors and Host Cells

In order to produce a disclosed recombinant Ate3C polypeptide, the DNA encoding the polypeptide can be chemically synthesized from published sequences or can be obtained directly from host cells harboring the gene (e.g., by cDNA library screening or PCR amplification). In some embodiments, the Ate3C polynucleotide is included in an expression cassette and/or cloned into a suitable expression vector by standard molecular cloning techniques. Such expression cassettes or vectors contain sequences that assist initiation and termination of transcription (e.g., promoters and terminators), and typically can also contain one or more selectable markers.

The expression cassette or vector is introduced into a suitable expression host cell, which then expresses the corresponding Ate3C polynucleotide. Suitable expression hosts may be bacterial or fungal microbes. Bacterial expression host may be, for example, Escherichia (e.g., Escherichia coli), Pseudomonas (e.g., P. fluorescens or P. stutzerei), Proteus (e.g., Proteus mirabilis), Ralstonia (e.g., Ralstonia eutropha), Streptomyces, Staphylococcus (e.g., S. carnosus), Lactococcus (e.g., L. lactis), or Bacillus (e.g., Bacillus subtilis, Bacillus megaterium, Bacillus licheniformis, etc.). Fungal expression hosts may be, for example, yeasts, which can also serve as ethanologens. Yeast expression hosts may be, for example, Saccharomyces cerevisiae, Schizosaccharomyces pombe, Yarrowia lipolytica, Hansenula polymorpha, Kluyveromyces lactis or Pichia pastoris. Fungal expression hosts may also be, for example, filamentous fungal hosts including Aspergillus niger, Chrysosporium lucknowense, Aspergillus (e.g., A. oryzae, A. niger, A. nidulans, etc.), Myceliophthora thermopile, or Trichoderma reesei. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing the Ate3C polypeptide.

Promoters and/or signal sequences associated with secreted proteins in a particular host of interest are candidates for use in the heterologous production and secretion of Ate3C polypeptides in that host or in other hosts. As an example, in filamentous fungal systems, the promoters that drive the genes for cellobiohydrolase I (cbhl), glucoamylase A (glaA), TAKA-amylase (amyA), xylanase (ex1A), the gpd-promoter cbhl, cbh1, endoglucanase genes eg1-eg5, Ce161B, Ce174A, gpd promoter, Pgk1, pki1, EF-1alpha, tef1, cDNA1 and hex1 are suitable and can be derived from a number of different organisms (e.g., A. niger, T. reesei, A. oryzae, A. awamori, A. nidulans).

In some embodiments, the Ate3C polynucleotide is recombinantly associated with a polynucleotide encoding a suitable homologous or heterologous signal sequence that leads to secretion of the recombinant Ate3C polypeptide into the extracellular (or periplasmic) space, thereby allowing direct detection of enzyme activity in the cell supernatant (or periplasmic space or lysate). Suitable signal sequences for Escherichia coli, other Gram negative bacteria and other organisms known in the art include those that drive expression of the HlyA, DsbA, Pbp, PhoA, PelB, OmpA, OmpT or M13 phage Gill genes. For Bacillus subtilis , Gram-positive organisms and other organisms known in the art, suitable signal sequences further include those that drive expression of the AprE, NprB, Mpr, AmyA, AmyE, Blac, SacB, and for S. cerevisiae or other yeast, including the killer toxin, Bar1, Suc2, Mating factor alpha, Inu1A or Ggplp signal sequence. Signal sequences can be cleaved by a number of signal peptidases, thus removing them from the rest of the expressed protein. Fungal expression signal sequences may be one that is selected from, for example, SEQ ID NOs: 13-37, herein. Yeast expression signal sequences may be one that is selected from, for example, SEQ ID NOs:38-40. Signal sequences that might be suitable for use to express the Ate3C polypeptides of the invention in Zymomonas mobilis may include, for example, one selected from SEQ ID NOs:41-42. (Linger, J. G. et al., (2010) Appl. Environ. Microbiol. 76 (19), 6360-6369).

In some embodiments, the recombinant Ate3C polypeptide is expressed alone or as a fusion with other peptides, tags or proteins located at the N- or C-terminus (e.g., 6XHis, HA or FLAG tags). Suitable fusions include tags, peptides or proteins that facilitate affinity purification or detection (e.g., 6XHis, HA, chitin binding protein, thioredoxin or FLAG tags), as well as those that facilitate expression, secretion or processing of the target beta-glucosidases Suitable processing sites include enterokinase, STE13, Kex2 or other protease cleavage sites for cleavage in vivo or in vitro.

Ate3C polynucleotides are introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

D. Cell Culture Media

Generally, the microorganism is cultivated in a cell culture medium suitable for production of the Ate3C polypeptides described herein. The cultivation takes place in a suitable nutrient medium comprising carbon and nitrogen sources and inorganic salts, using procedures and variations known in the art. Suitable culture media, temperature ranges and other conditions for growth and cellulase production are known in the art. As a non-limiting example, a typical temperature range for the production of cellulases by Trichoderma reesei is 24° C. to 37° C., for example, between 25° C. and 30° C.

1. Cell Culture Conditions

Materials and methods suitable for the maintenance and growth of fungal cultures are well known in the art. In some aspects, the cells are cultured in a culture medium under conditions permitting the expression of one or more beta-glucosidase polypeptides encoded by a nucleic acid inserted into the host cells. Standard cell culture conditions can be used to culture the cells. In some aspects, cells are grown and maintained at an appropriate temperature, gas mixture, and pH. In some aspects, cells are grown at in an appropriate cell medium.

### IV. Activities of Ate3C

The recombinant Ate3C polypeptides disclosed herein have beta-glucosidase activity or a capacity to hydrolyze cellobiose and liberating D-glucose therefrom. The Ate3C polypeptides herein have higher beta-glucosidase activity and improved or increased capacity to liberate D-glucose from cellobiose than the benchmark high fidelity beta-glucosidase Bgl1 of Trichoderma reesei, under the same saccharification conditions. In some embodiments, the Ate3C polypeptides herein have higher beta-glucosidase activity and/or improved or increased capacity to liberate D-glucose from cellobiose than another benchmark beta-glucosidase B-glu of Aspergillus niger.

As shown in Examples 3, the recombinant Ate3C polypeptide, as compared to the Trichoderma reesei Bgl1, has at least 10%, preferably at least 20%, more preferably at least 30% less activity hydrolyzing a chloro-nitro-phenyl-glucoside (CNPG) substrate. In some embodiments, the recombinant Ate3C polypeptide, as compared to the Aspergillus niger B-glu, has at least the same, 1.5-fold, 2-fold, 3-fold, 4 fold, or even 5-fold higher activity hydrolyzing a CNPG substrate.

The recombinant Ate3C polypeptide, as compared to the Trichoderma reesei Bgl1, has dramatically improved or increased, for example, at least 30% higher, more preferably at least 50% higher, preferably at least 60% higher, more preferably at least 80% higher, preferably at least 90%, even more preferably at least 100% higher, and most preferably at least 120% higher cellobiase activity, which measures the enzymes' capability to catalyze the hydrolysis of cellobiose, liberating D-glucose. In some embodiments, the recombinant Ate3C polypeptide, as compared to the Aspergillus niger B-glu, has about ½, about ⅓, about ¼, or even about ⅕ of the capability to catalyze the hydrolysis of cellobiose, liberating D-glucose.

In some embodiments, the recombinant Ate3C polypeptide, as compared to the Trichoderma reesei Bgl1, has about 2-fold, about 3-fold, about 4-fold, or even about 5-fold lower hydrolysis activity ratio over CNPG/cellobiose. In some embodiments, the Ate3C polypeptide, as compared to the Aspergillus niger B-glu, has about 2-fold, about 3-fold, about 4-fold, about 5-fold, or even about 6-fold higher relative hydrolysis activity ratio over CNPG/cellobiose.

As shown in Example 4, the recombinant Ate3C polypeptide, as compared to the Trichoderma reesei Bgl1, produced more glucose but equal or less amount of total sugars from a phosphoric acid swollen cellulose substrate.

As shown in Example 5, the recombinant Ate3C polypeptide, as compared to the Trichoderma reesei Bgl1, also produced more glucose but equal or less amount of total sugars from a dilute ammonia pretreated corn stover substrate.

### V. Compositions Comprising a Recombinant Beta-Glucosidase Ate3C Polypeptide

The present disclosure provides engineered enzyme compositions (e.g., cellulase compositions) or fermentation broths enriched with a recombinant Ate3C polypeptides. In some aspects, the composition is a cellulase composition. The cellulase composition can be, e.g., a filamentous fungal cellulase composition, such as a Trichoderma cellulase composition. In some aspects, the composition is a cell comprising one or more nucleic acids encoding one or more cellulase polypeptides. In some aspects, the composition is a fermentation broth comprising cellulase activity, wherein the broth is capable of converting greater than about 50% by weight of the cellulose present in a biomass sample into sugars. The term “fermentation broth” and “whole broth” as used herein refers to an enzyme preparation produced by fermentation of an engineered microorganism that undergoes no or minimal recovery and/or purification subsequent to fermentation. The fermentation broth can be a fermentation broth of a filamentous fungus, for example, a Trichoderma, Humicola, Fusarium, Aspergillus, Neurospora, Penicillium, Cephalosporium, Achlya, Podospora, Endothia, Mucor, Cochliobolus, Pyricularia, Myceliophthora or Chrysosporium fermentation broth. In particular, the fermentation broth can be, for example, one of Trichoderma spp. such as a Trichoderma reesei, or Penicillium spp., such as a Penicillium funiculosum. The fermentation broth can also suitably be a cell-free fermentation broth. In one aspect, any of the cellulase, cell, or fermentation broth compositions of the present invention can further comprise one or more hemicellulases.

In some aspects, the whole broth composition is expressed in T. reesei or an engineered strain thereof. In some aspects the whole broth is expressed in an integrated strain of T. reesei wherein a number of cellulases including an Ate3C polypeptide has been integrated into the genome of the T. reesei host cell. In some aspects, one or more components of the polypeptides expressed in the integrated T. reesei strain have been deleted.

In some aspects, the whole broth composition is expressed in A. niger or an engineered strain thereof.

Alternatively, the recombinant Ate3C polypeptides can be expressed intracellularly. Optionally, after intracellular expression of the enzyme variants, or secretion into the periplasmic space using signal sequences such as those mentioned above, a permeabilization or lysis step can be used to release the recombinant Ate3C polypeptide into the supernatant. The disruption of the membrane barrier is effected by the use of mechanical means such as ultrasonic waves, pressure treatment (French press), cavitation, or by the use of membrane-digesting enzymes such as lysozyme or enzyme mixtures. A variation of this embodiment includes the expression of a recombinant Ate3C polypeptide in an ethanologen microbe intracellularly. For example, a cellobiose transporter can be introduced through genetic engineering into the same ethanologen microbe such that cellobiose resulting from the hydrolysis of a lignocellulosic biomass can be transported into the ethanologen organism, and can therein be hydrolyzed and turned into D-glucose, which can in turn be metabolized by the ethanologen.

In some aspects, the polynucleotides encoding the recombinant Ate3C polypeptide are expressed using a suitable cell-free expression system. In cell-free systems, the polynucleotide of interest is typically transcribed with the assistance of a promoter, but ligation to form a circular expression vector is optional. In some embodiments, RNA is exogenously added or generated without transcription and translated in cell-free systems.

### VI. Uses of Ate3C Polypeptides to Hydrolyze a Lignocellulosic Biomass Substrate

In some aspects, provided herein are methods for converting lignocelluloses biomass to sugars, the method comprising contacting the biomass substrate with a composition disclosed herein comprising an Ate3C polypeptide in an amount effective to convert the biomass substrate to fermentable sugars. In some aspects, the method further comprises pretreating the biomass with acid and/or base and/or mechanical or other physical means In some aspects the acid comprises phosphoric acid. In some aspects, the base comprises sodium hydroxide or ammonia. In some aspects, the mechanical means may include, for example, pulling, pressing, crushing, grinding, and other means of physically breaking down the lignocellulosic biomass into smaller physical forms. Other physical means may also include, for example, using steam or other pressurized fume or vapor to “loosen” the lignocellulosic biomass in order to increase accessibility by the enzymes to the cellulose and hemicellulose. In certain embodiments, the method of pretreatment may also involve enzymes that are capable of breaking down the lignin of the lignocellulosic biomass substrate, such that the accessibility of the enzymes of the biomass hydrolyzing enzyme composition to the cellulose and the hemicelluloses of the biomass is increased.

Biomass: The disclosure provides methods and processes for biomass saccharification, using the enzyme compositions of the disclosure, comprising an Ate3C polypeptide. The term “biomass,” as used herein, refers to any composition comprising cellulose and/or hemicellulose (optionally also lignin in lignocellulosic biomass materials). As used herein, biomass includes, without limitation, seeds, grains, tubers, plant waste (such as, for example, empty fruit bunches of the palm trees, or palm fiber wastes) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure therefore provides methods of saccharification comprising contacting a composition comprising a biomass material, for example, a material comprising xylan, hemicellulose, cellulose, and/or a fermentable sugar, with an Ate3C polypeptide of the disclosure, or an Ate3C polypeptide encoded by a nucleic acid or polynucleotide of the disclosure, or any one of the cellulase or non-naturally occurring hemicellulase compositions comprising an Ate3C polypeptide, or products of manufacture of the disclosure.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Pretreatment: Prior to saccharification or enzymatic hydrolysis and/or fermentation of the fermentable sugars resulting from the saccharification, biomass (e.g., lignocellulosic material) is preferably subject to one or more pretreatment step(s) in order to render xylan, hemicellulose, cellulose and/or lignin material more accessible or susceptible to the enzymes in the enzymatic composition (for example, the enzymatic composition of the present invention comprising an Ate3C polypeptide) and thus more amenable to hydrolysis by the enzyme(s) and/or the enzyme compositions.

In some aspects, a suitable pretreatment method may involve subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

In some aspects, a suitable pretreatment method may involve subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

In further aspects, a suitable pretreatment method may involve processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

In yet further aspects, a suitable pretreatment method may involve pre-hydrolyzing biomass (e.g., lignocellulosic materials) in a pre-hydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. In a variation of this aspect, the pre-hydrolyzing can alternatively or further involves pre-hydrolysis using enzymes that are, for example, capable of breaking down the lignin of the lignocellulosic biomass material.

In yet further aspects, suitable pretreatments may involve the use of hydrogen peroxide H₂O₂. See Gould, 1984, Biotech, and Bioengr. 26:46-52.

In other aspects, pretreatment can also comprise contacting a biomass material with stoichiometric amounts of sodium hydroxide and ammonium hydroxide at a very low concentration. See Teixeira et al., (1999), Appl. Biochem.and Biotech. 77-79:19-34.

In some embodiments, pretreatment can comprise contacting a lignocellulose with a chemical (e.g., a base, such as sodium carbonate or potassium hydroxide) at a pH of about 9 to about 14 at moderate temperature, pressure, and pH. See Published International Application WO2004/081185. Ammonia is used, for example, in a preferred pretreatment method. Such a pretreatment method comprises subjecting a biomass material to low ammonia concentration under conditions of high solids. See, e.g., U.S. Patent Publication No. 20070031918 and Published International Application WO 06110901.

A. The Saccharification Process

In some aspects, provided herein is a saccharification process comprising treating biomass with an enzyme composition comprising a polypeptide, wherein the polypeptide has beta-glucosidase activity and wherein the process results in at least about 50 wt.% (e.g., at least about 55 wt.%, 60 wt.%, 65 wt.%, 70 wt.%, 75 wt.%, or 80 wt.%) conversion of biomass to fermentable sugars. In some aspects, the biomass comprises lignin. In some aspects the biomass comprises cellulose. In some aspects the biomass comprises hemicellulose. In some aspects, the biomass comprising cellulose further comprises one or more of xylan, galactan, or arabinan. In some aspects, the biomass may be, without limitation, seeds, grains, tubers, plant waste (e.g., empty fruit bunch from palm trees, or palm fiber waste) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like), potatoes, soybean (e.g., rapeseed), barley, rye, oats, wheat, beets, and sugar cane bagasse. In some aspects, the material comprising biomass is subject to one or more pretreatment methods/steps prior to treatment with the polypeptide. In some aspects, the saccharification or enzymatic hydrolysis further comprises treating the biomass with an enzyme composition comprising an Ate3C polypeptide of the invention. The enzyme composition may, for example, comprise one or more other cellulases, in addition to the Ate3C polypeptide. Alternatively, the enzyme composition may comprise one or more other hemicellulases. In certain embodiments, the enzyme composition comprises an Ate3C polypeptide of the invention, one or more other cellulases, one or more hemicellulases. In some embodiments, the enzyme composition is a whole broth composition.

In some aspects, provided is a saccharification process comprising treating a lignocellulosic biomass material with a composition comprising a polypeptide, wherein the polypeptide has at least about 85% (e.g., at least about 85%, 90%, 91%, 92%, 93%, 94%, 95%, 96%, 97%, 98%, 99%) sequence identity to SEQ ID NO:2, and wherein the process results in at least about 50% (e.g., at least about 55%, 60%, 65%, 70%, 75%, 80%, 85%, or 90%) by weight conversion of biomass to fermentable sugars. In some aspects, lignocellulosic biomass material has been subject to one or more pretreatment methods/steps as described herein.

Other aspects and embodiments of the present compositions and methods will be apparent from the foregoing description and following examples.

### EXAMPLES

The following examples are provided to demonstrate and illustrate certain preferred embodiments and aspects of the present disclosure and should not be construed as limiting.

### Examples 1

1-A. Cloning & Expression of Gene Expression of Ate3C and Benchmark T. reesei Bgl1.

1-A-a. Construction of the T. reesei bgl1 Expression Vector

The N-terminal portion of the native T. reesei β-glucosidase gene bgl1 was codon optimized (DNA 2.0, Menlo Park, Calif.). This synthesized portion comprised the first 447 bases of the coding region of this enzyme. This fragment was then amplified by PCR using primers SK943 and SK941 (below). The remaining region of the native bgl1 gene was PCR amplified from a genomic DNA sample extracted from T. reesei strain RL-P37 (Sheir-Neiss, G et al. (1984) Appl. Microbiol. Biotechnol. 20:46-53), using the primers SK940 and SK942 (below). These two PCR fragments of the bgl1 gene were fused together in a fusion PCR reaction, using primers SK943 and SK942:

Forward Primer SK943:

(SEQ ID NO: 5)

(5′-CACCATGAGATATAGAACAGCTGCCGCT-3′)
Reverse Primer SK941:

(SEQ ID NO: 6)

(5′-CGACCGCCCTGCGGAGTCTTGCCCAGTGGTCCCGCGACAG-3′)
Forward Primer (SK940):

(SEQ ID NO: 7)

(5′-CTGTCGCGGGACCACTGGGCAAGACTCCGCAGGGCGGTCG-3′)
Reverse Primer (SK942):

(SEQ ID NO: 8)

(5′-CCTACGCTACCGACAGAGTG-3′)

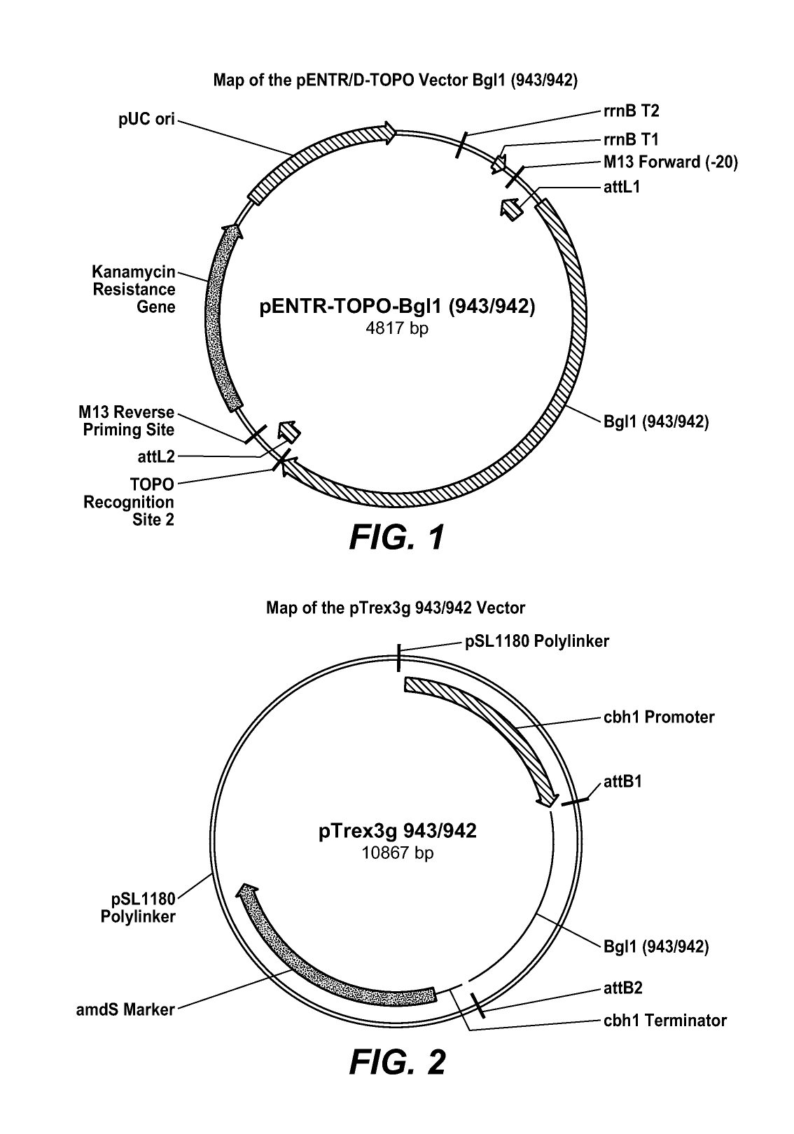

The resulting fusion PCR fragments were cloned into the Gateway® Entry vector pENTR™/D-TOPO® (FIG. 1), and transformed into E. coli One Shot® TOP10 Chemically Competent cells (Invitrogen) resulting in the intermediate vector, pENTR TOPO-Bgl1(943/942) (FIG. 1). The nucleotide sequence of the inserted DNA was determined. The pENTR-943/942 vector with the correct bgl1 sequence was recombined with pTrex3g using a LR clonase® reaction (see, protocols outlined by Invitrogen). The LR clonase reaction mixture was transformed into E. coli One Shot®TOP10 Chemically Competent cells (Invitrogen), resulting in the expression vector, pTrex3g 943/942 (FIG. 2). The vector also contained the Aspergillus nidulans amdS gene, encoding acetamidase, as a selectable marker for transformation of T. reesei. The expression cassette was PCR amplified with primers SK745 and SK771 (below) to generate the product for transformation.

Forward Primer SK771:

(SEQ ID NO: 9)

(5′-GTCTAGACTGGAAACGCAAC-3′)
Reverse Primer SK745:

(SEQ ID NO: 10)

(5′-GAGTTGTGAAGTCGGTAATCC-3′)

1-A-b. Construction of the Ate3C Expression Vector

The open reading frame of the beta-glucosidase gene was amplified by PCR using genomic DNA extracted from Aspergillus terreus as the template. The open reading frame was amplified with the native signal sequence. The PCR thermocycler used was DNA Engine Tetrad 2 Peltier Thermal Cycler (BioRad Laboratories). The DNA polymerase used was PfuUltra II Fusion HS DNA Polymerase (Stratagene) or a similar quality proofreading DNA polymerase. The primers used to amplify the open reading frame were as follows:

Ate3C-F:

(SEQ ID NO: 11)

5′-CAC CAT GAA GCT TTC CAT TTT GGA GGC AGC-3′
Ate3C-R:

(SEQ ID NO: 12)

5′-TTA CTG CAC CCG TGG CAA GCG A-3′

The Ate3C-F forward primer included four additional nucleotides (sequences-CACC) at the 5′-end to facilitate directional cloning into pENTR/D-TOPO. The PCR product of the open reading frame was purified using a Qiaquick PCR Purification Kit (Qiagen, Valencia, Calif.). The purified PCR product was cloned into the pENTR/D-TOPO vector (Invitrogen), transformed into TOP10 chemically competent E. coli cells (Invitrogen, Carlsbad, Calif.) and plated on LA plates with 50 ppm kanamycin. Plasmid DNA was obtained from the E. coli transformants using a QlAspin plasmid preparation kit (Qiagen).

Sequence data for the DNA inserted in the pENTR/D-TOPO vector was obtained using M13 forward and reverse primers. A pENTR/D-TOPO vector with the correct DNA sequence of the open reading frame was recombined with the pTrex3gM destination vector (FIG. 2) using LR clonase reaction mixture (Invitrogen, Carlsbad, Calif.) according to the manufacturer's instructions.

The product of the LR clonase reaction was subsequently transformed into TOP10 chemically competent E. coli cells which were then plated on LA containing 50 ppm carbenicillin. The resulting pExpression construct was pTrex3gM containing the Ate3C open reading frame and the Aspergillus tubingensis acetamidase selection marker (amdS). DNA of the pExpression construct was isolated using a Qiagen miniprep kit and used for transformation of Trichoderma reesei.

Either the pExpression plasmid or a PCR product of the expression cassette was transformed into a T. reesei six-fold-delete strain see, e.g., the description in International Patent Application Publication No. WO 2010/141779) using the PEG-mediated protoplast method with slight modifications as described below. For protoplast preparation, spores were grown for 16-24 h at 24° C. in Trichoderma Minimal Medium MM, which contained 20 g/L glucose, 15 g/L KH₂PO₄, pH 4.5, 5 g/L (NH₄)₂SO_{4,} 0.6 g/L MgSO_{4×}7H₂O, 0.6 g/L CaCl_{2×}2H₂O, 1 mL of 1000 ×T. reesei Trace elements solution (which contained 5 g/L FeSO₄×7H₂O, 1.4 g/L ZnSO₄×7H₂O, 1.6 g/L MnSO₄×H₂O, 3.7 g/L CoCl₂×6H₂O) with shaking at 150 rpm. Germinating spores were harvested by centrifugation and treated with 50 mg/mL of Glucanex G200 (Novozymes AG) solution to lyse the fungal cell walls. Further preparation of the protoplasts was performed in accordance with a method described by Penttila et al. (1987) Gene 61: 155-164. The transformation mixtures, which contained about 1 μg of DNA and 1-5×10⁷ protoplasts in a total volume of 200 μL, were each treated with 2 mL of 25% PEG solution, diluted with 2 volumes of 1.2 M sorbitol/10 mM Tris, pH7.5, 10 mM CaCl₂, mixed with 3% selective top agarose MM containing 5 mM uridine and 20 mM acetamide. The resulting mixtures were poured onto 2% selective agarose plate containing uridine and acetamide. Plates were incubated further for 7-10 d at 28° C. before single transformants were picked onto fresh MM plates containing uridine and acetamide. Spores from independent clones were used to inoculate a fermentation medium in shake flasks.

The fermentation media was 36 mL of defined broth containing glucose/sophorose and 2 g/L uridine, such as Glycine Minimal media (6.0 g/L glycine; 4.7 g/L (NH₄)₂SO₄; 5.0 g/L KH₂PO₄; 1.0 g/L MgSO₄.7H₂O; 33.0 g/L PIPPS; pH 5.5) with post sterile addition of ˜2% glucose/sophorose mixture as the carbon source, 10 ml/L of 100 g/L of CaCl₂, 2.5 ml/L of T. reesei trace elements (400×) : 175 g/L Citric acid anhydrous; 200 g/L FeSO₄.7H₂O; 16 g/L ZnSO₄·7H₂O; 3.2 g/L CuSO₄.5H₂O; 1.4 g/L MnSO₄.H₂O; 0.8 g/L H₃BO₃, in 250 ml Thomson Ultra Yield Flasks (Thomson Instrument Co., Oceanside, Calif.).

1-A-c. Construction of a Yeast Shuttle Vector pSC11

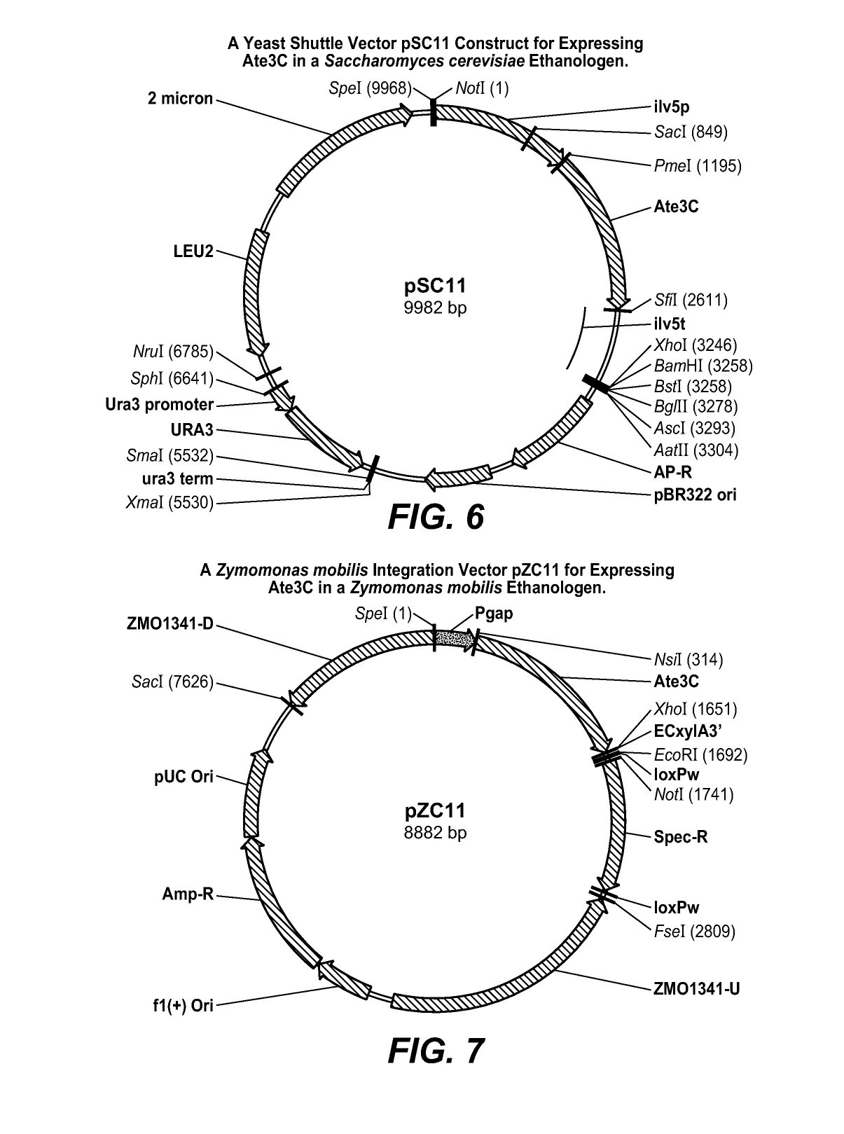

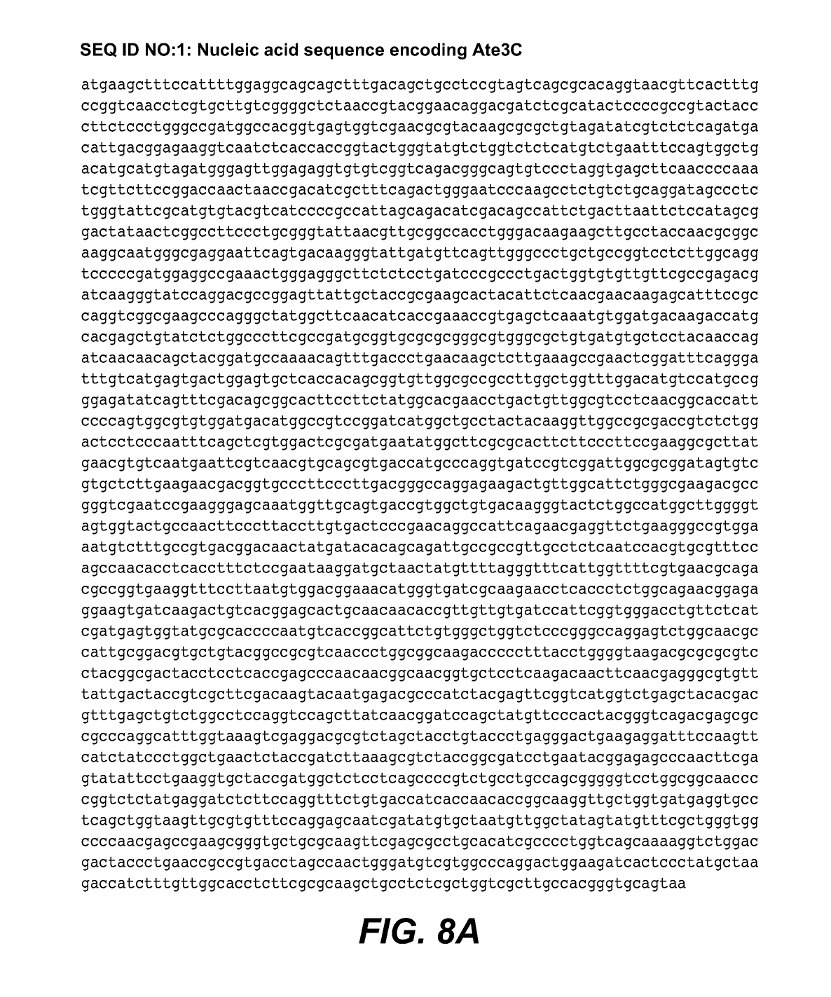

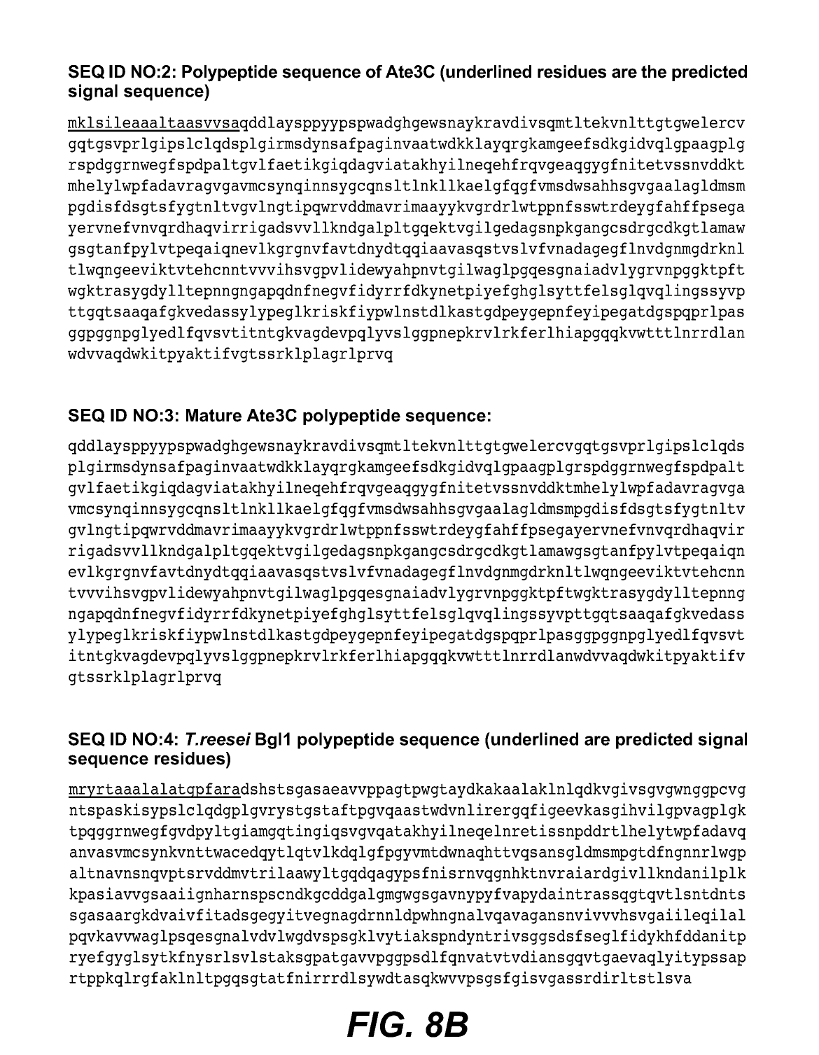

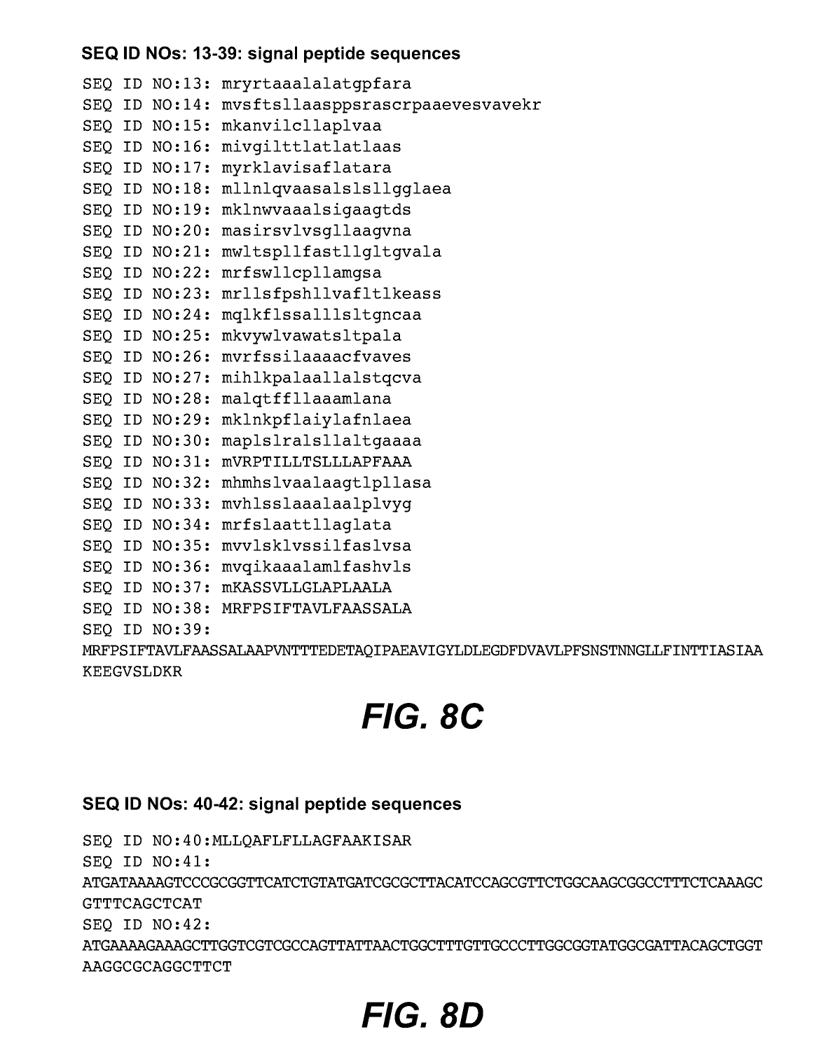

A yeast shuttle vector can be constructed in accordance with the vector map of FIG. 6. This vector can be used to express an Ate3C polypeptide in Saccharomyces cerevisiae intracellularly. A cellobiose transporter can be introduced into the Saccharomyces cerevisiae in the same shuttle vector or in a separate vector using known methods, such as, for example, those described by Ha et al., (2011) in PNAS, 108(2): 504-509.

Transformation of expression cassettes can be performed using the yeast EZ-Transformation kit. Transformants can be selected using YSC medium, which contains 20 g/L cellobiose. The successful introduction of the expression cassettes into yeast can be confirmed by colony PCR with specific primers.

Yeast strains can be cultivated in accordance with known methods and protocols. For example, they can be cultivated at 30° C. in a YP medium (10 g/L yeast extract, 20 g/L Bacto peptone) with 20 g/L glucose. To select transformants using an amino acid auxotrophic marker, yeast synthetic complete (YSC) medium may be used, which contains 6.7 g/L yeast nitrogen base plus 20 g/L glucose, 20 g/L agar, and CSM-Leu-Trp-Ura to supply nucleotides and amino acids.

1-A-d. Construction of a Zymomonas mobilis Integration Vector pZC11.

A Zymomonas mobilis integration vector pZC11 can be constructed in accordance with the vector map of FIG. 7. This vector can be used to express an Ate3C polypeptide in Zymomonas mobilis intracellularly. A cellobiose transporter can be introduced into the Zymomonas mobilis in the same integration vector or in a separate vector using known methods of introducing those transporters into a bacterial cell, such as, for example, those described by Sekar et al., (2012) Applied Environmental Microbiology, 78(5):1611-1614et al.

Successful introduction of the integration vector as well as the cellobiose transporter gene can be confirmed using various known approaches, for example by PCR using confirmatory primers specifically designed for this purpose.

Zymomonas mobilis strains can be cultivated and fermented according to known methods, such as, for example, those described in U.S. Pat. No. 7,741,119.

1-B. Purification of T. reesei BGl1 & Ate3C

T. reesei Bgl1 was over-expressed in, and purified from, the fermentation broth of a six-fold-deleted Trichoderma reesei host strain (see, e.g., the description in Published International Patent Application Publication No. WO 2010/141779). A concentrated broth was loaded onto a G25 SEC column (GE Healthcase Bio-Sciences) and was buffer-exchanged against 50 mM sodium acetate, pH 5.0. The buffer exchanged Bgl1 was then loaded onto a 25 mL column packed with amino benzyl-S-glucopyranosyl sepharose affinity matrix. After extensive washing with 250 mM sodium chloride in 50 mM sodium acetate, pH 5.0, the bound fraction was eluted with 100 mM glucose in 50 mM sodium acetate and 250 mM sodium chloride, pH 5.0. The eluted fractions that tested positive for chloro-nitro-phenyl glucoside (CNPG) activity were pooled and concentrated. A single band corresponding to the MW of the T. reesei Bgl1 on SDS-PAGE and confirmed by mass spectrometry verified the purity of the eluted Bgl1. The final stock concentration was determined to be 2.2 mg/mL by absorbance at 280 nm.

An Ate3C expressed by Tricoderma reesei as described above can be purified from a concentrated fermentation broth by first diluting 100 mg into a 50 mM MES buffer, pH 6.0. The Ate3C can then be enriched by loading 2 mg protein per mL resin onto a SP Sepharose ion exchange resin (GE Healthcare) charged at pH 6. The Ate3C can be eluted in the flow-through. The enriched Ate3C can then be concentrated using a 10,000 MW cut-off membrane (Vivaspin, GE Healthcare) to a volume 5 times lower from the original volume. The other background components can be removed from Ate3C by adding 40% ammonium sulfate (w/v) in batch mode. Pure Mg3A can be recovered on the supernatant after centrifugation. Ate3C is then simultaneously dialyzed and concentrated in 50 mM MES buffer, pH 6.0, using a 10,000 MW cut off membrane (Vivaspin, GE Healthcare). The activity and purity of Ate3C can be assessed by the chloro-nitro-phenyl glucoside assay and SDS-PAGE, respectively. The supernatant can then be dialyzed extensively against 50 mM MES, 100 mM NaCl buffer, pH 6.0 using a 7,000 MW cut off membrane dialysis cassette (PIERCE). The activity of the final Ate3C batch can be determined by chloro-nitro-phenyl glucoside assay. The concentration can be determined by the bicinchoninic acid assay (PIERCE) and by the absorbance assay at a wavelength of 280 nm using a molar extinction coefficient calculated by GPMAW v 7.0.

### Example 2:

### Various Assays

### 2-A. Protein Concentration Measurement by UPLC

An Agilent HPLC 1290 Infinity system was used for protein quantitation with a Waters ACQUITY UPLC BEH C4 Column (1.7 μm, 1×50 mm). A six minute program with an initial gradient from 5% to 33% acetonitrile (Sigma-Aldrich) in 0.5 min, followed by a gradient from 33% to 48% in 4.5 min, and then a step gradient to 90% acetronitrile was used. A protein standard curve based on the purified Trichoderma reesei Bgl1 was used to quantify the Ate3C polypeptides.

### 2-B. Chloro-nitro-phenyl-glucoside (CNPG) Hydrolysis Assay

Two hundred (200) μL of a 50 mM sodium acetate buffer, pH 5 was added to individual wells of a microtiter plate. Five (5) μL of enzyme, diluted in 50 mM sodium acetate buffer, pH 5, was also added to individual wells. The plate was covered and allowed to equilibrate at 37° C. for 15 min in an Eppendorf Thermomixer. Twenty (20) μL of 2 mM 2-Chloro-4-nitrophenyl-beta-D-Glucopyranoside (CNPG, Rose Scientific Ltd., Edmonton, Calif.) prepared in Millipore water was added to individual wells and the plate was quickly transferred to a spectrophotometer (SpectraMax 250, Molecular Devices). A kinetic read was performed at OD 405 nm for 15 min and the data recorded as Vmax. The extinction coefficient for CNP was used to convert Vmax from units of OD/sec to μM CNP/sec. Specific activity (μM CNP/sec/mg Protein) was determined by dividing μM CNP/sec by the mg of enzyme used in the assay. Standard error for the CNPG assay was determined to be 3%.

### 2-C. Cellobiose Hydrolysis Assay

Cellobiase activity was determined at 50° C. using the method of Ghose, T. K. Pure & Applied Chemistry, 1987, 59 (2), 257-268. Cellobiose units (derived as described in Ghose) are defined as 0.0926 divided by the amount of enzyme required to release 0.1 mg glucose under the assay conditions. Standard error for the cellobiase assay was determined to be 10%.

### 2-D. Preparation of Phosphoric Acid Swollen Cellulose (PASC)

Phosphoric acid swollen cellulose (PASC) was prepared from Avicel using an adapted protocol of Walseth, TAPPI 1971, 35:228 and Wood, Biochem. J. 1971, 121:353-362. In short, Avicel PH-101 was solubilized in concentrated phosphoric acid then precipitated using cold deionized water. The cellulose was collected and washed with more water to neutralize the pH. It was diluted to 1% solids in 50 mM sodium acetate pH5.

### Example 3.

### Improved Hydrolysis Performance of Ate3C Over the Benchmark Trichoderma reesei Bgl1, or Over the Benchmark Aspergillus niger B-glu, as Seen in CNPG and Cellobiase Assays.

### 3-A. CNPG and Cellobiase Activity of Beta-Glucosidases Produced in Shake Flask

The concentration of Ate3C in the crude shake flask broth was measured by UPLC (described herein) and determined to be 0.116 g/L. Two cellobiohydrolases were included in the following experiments as controls for beta-glucosidase activity in the expression strain background and were below the detection limit of the assays. Purified Trichoderma reesei Bgl1 was used from a stock of 2.2 mg/mL (A280 measurement). Purified A. niger Bglu was obtained from Megazyme International, without BSA (Megazyme International Ireland Ltd., Wicklow, Ireland, Lot No. 031809),

The activity of each enzyme on the model substrates chloro-nitro-phenyl-glucoside (CNPG) and cellobiose were measured. The assays were each carried out at the temperature in the standard protocol; CNPG at 37° C. and cellobiose at 50° C.

TABLE 3-1

Ratio to T. reesei Bgl1

| Enzyme | Purified | CNPG | Cellobiose |
|---|---|---|---|
| T. reesei Bgl | Y | 1 | 1 |
| A. niger B-glu | Y | 0.13 | 12 |
| Ate3C | N | 0.53 | 2.72 |

Ate3C had half the CNPG activity of Trichoderma reesei Bgl1, and more than two and a half times the cellobiose hydrolysis activity. A. niger beta-glucosidase, B-glu, had about one tenth the CNPG activity of Tricoderma reesei Bgl and twelve times the cellobiase activity. The cellobiohydrolases had no activity on cellobiose (no glucose was observed for any wells, data not shown). Thus, the six-delete host background did not contribute to the small molecule activity measurements.

TABLE 3-2

| Enzyme | Purified | CNPG/Cellobiase |
|---|---|---|
| T. reesei Bgl1 | Y | 62 |
| A. niger b-glu | Y | 1.6 |
| Ate3C | N | 12 |

To compare the activity of each molecule independent of protein determination, the ratio of CNPG to cellobiase activity was calculated. The ratio of CNPG/cellobiase activity for Ate3C was lower than for Trichoderma reesei Bgl1, by about 5-fold. The ratio of CNPG/cellobiase activity for Ate3C was, however, significantly higher than that of Aspergillus niger B-glu, by about 7-fold

### Example 4:

### Improved Hydrolysis Performance of Ate3C Polypeptides on PASC Substrates.

4-A. Dose Curves Depicting the Measurements and Comparison of Ate3C Vs. the Benchmark Trichoderma reesei Bgl1 Hydrolysis of PASC, in a Background Whole Cellulase Composition Produced by a Strain Described in International Published Patent Application No. WO 2011/038019.

The beta-glucosidases were added from 0-10 mg protein/g glucan to a constant loading of 10 mg protein/g glucan whole cellulase produced by a strain described in International Published Patent Application No. WO 2011/038019, which expresses Fv43D, Fv3A, Fv51A, AfuXyn2, EG4, and etc). The mixtures were used to hydrolyze phosphoric acid swollen cellulose (PASC). Each sample dose was assayed in quadruplicate.

All enzyme dilutions were made into 50 mM sodium acetate buffer, pH 5.0. One hundred and fifty (150) μL of cold 0.6% PASC was added to 30 μof enzyme solution in microtiter plates (NUNC flat bottom PS, cat. no. 269787). The enzyme mixture therefore contained 10 mg protein/g glucan of the whole cellulase plus 0-10 mg of Ate3C or Bgl1/g glucan. The plates were covered with aluminum plate seals and incubated for 1.5 h at 50° C., 200 rpm in an Innova incubator/shaker. The reaction was quenched with 100 μL of 100 mM Glycine, pH 10, filtered (Millipore vacuum filter plate cat. no. MAHVN45) and the soluble sugars were measured on an Agilent 5042-1385 HPLC with an Aminex HPX-87P column.

Percent glucan conversion was determined as (mg glucose+mg cellobiose+mg cellotriose)/mg cellulose in the reaction.

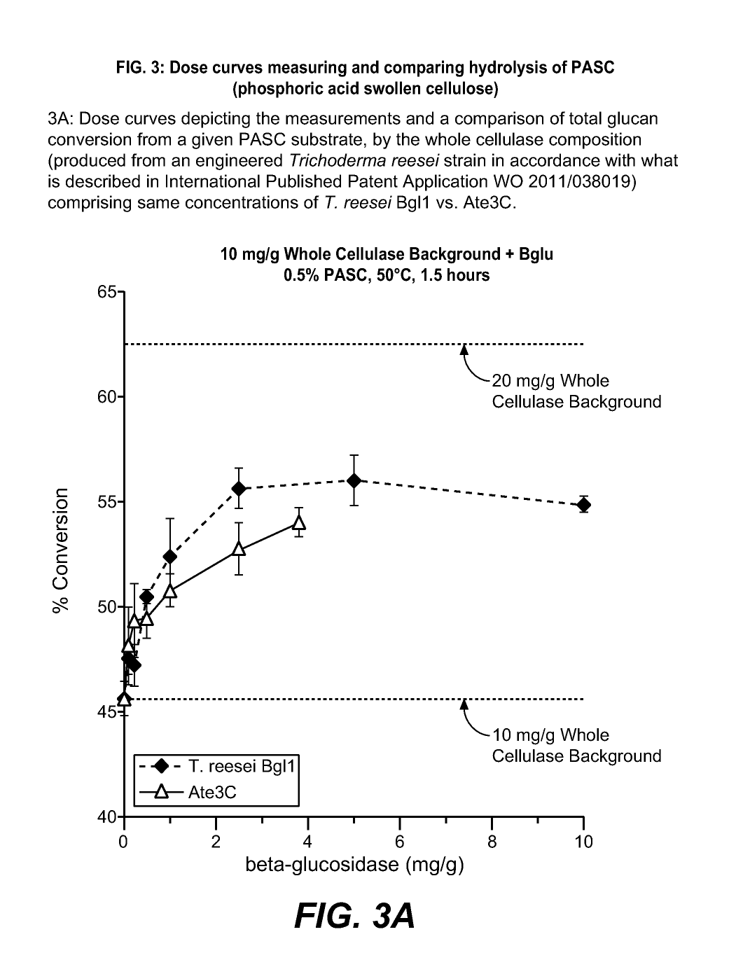

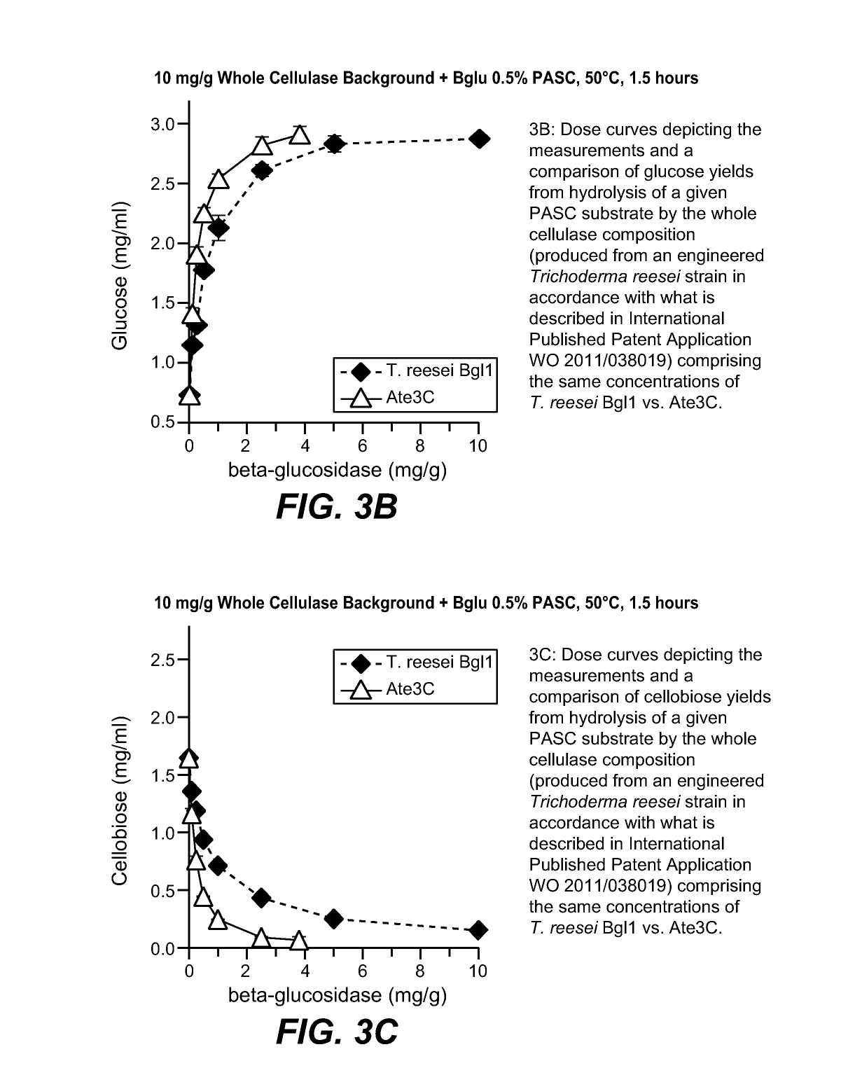

The results are shown in FIG. 3 (including the dose curves of 3A-3C).

Ate3C produced more glucose, but not more total sugar, than the same dose of T. reesei Bg11. This is consistent with Ate3C having higher cellobiase activity than T. reesei Bgl1.

### Example 5:

### Improved Hydrolysis Performance of Ate3C Polypeptides on Dilute Ammonia Pretreated Corn Stover (DACS) substrates.

5-A. Dose Curves Depicting the Measurements and Comparison of Ate3C Vs. the Benchmark Trichoderma reesei Bgl1 Hydrolysis of DACS, in a Background Whole Cellulase Composition Produced by a Strain Described in International Published Patent Application No. WO 2011/038019.

Shake flask produced Ate3C culture broth was concentrated >20 fold using 10,000 molecular weight cut off PES spin concentrators. Protein concentration was determined by UPLC, compared to a Trichoderma reesei Bgl1 standard curve. The concentrated Ate3C sample was used in saccharification assays with dilute ammonia pretreated corn stover (DACS). Each enzyme mixture sample was blended with 10% of Ate3C or Bgl1, with a whole cellulase produced by an engineered Trichoderma reesei strain as described in International Published Patent Application No. WO 2011/038019. Dose response curves for hydrolyzing the DACS substrate were generated by adding 3-53 mg total protein/g glucan enzyme mixture to the substrate.

Trichoderma reesei Bgl1 was added to the hydrolysis assay from a purified stock of 2.2 g/L total protein. Ate3C was added from the 2.89 g/L concentrated sample.

Dilute ammonia pre-treated corn stover (DACS) was slurried in 20 mM Sodium Acetate, pH 5 for a final 7% glucan (21.5% solids) content. If needed, the slurry was adjusted to pH 5 and the slurry was transferred into 96-well microtiter plates.

All enzymes were loaded based on mg protein/g glucan in the substrate. All enzyme dilutions were made into 50 mM Sodium Acetate buffer, pH 5.0. Thirty (30) μL of enzyme solution and 45 μg DACS substrate were added per well in 96-well microtiter plates. Each sample dose was tested in quadruplicate. The plates were covered with an aluminum seal and incubated for 2 days at 50° C., 200 rpm in the Innova incubator/shaker. The reaction was quenched with 100 μL of 100 mM Glycine, pH 10, filtered, and the soluble sugars were measured by HPLC.

Percent glucan conversion is defined as (mg glucose+mg cellobiose+mg cellotriose)/mg cellulose in the DACS substrate.

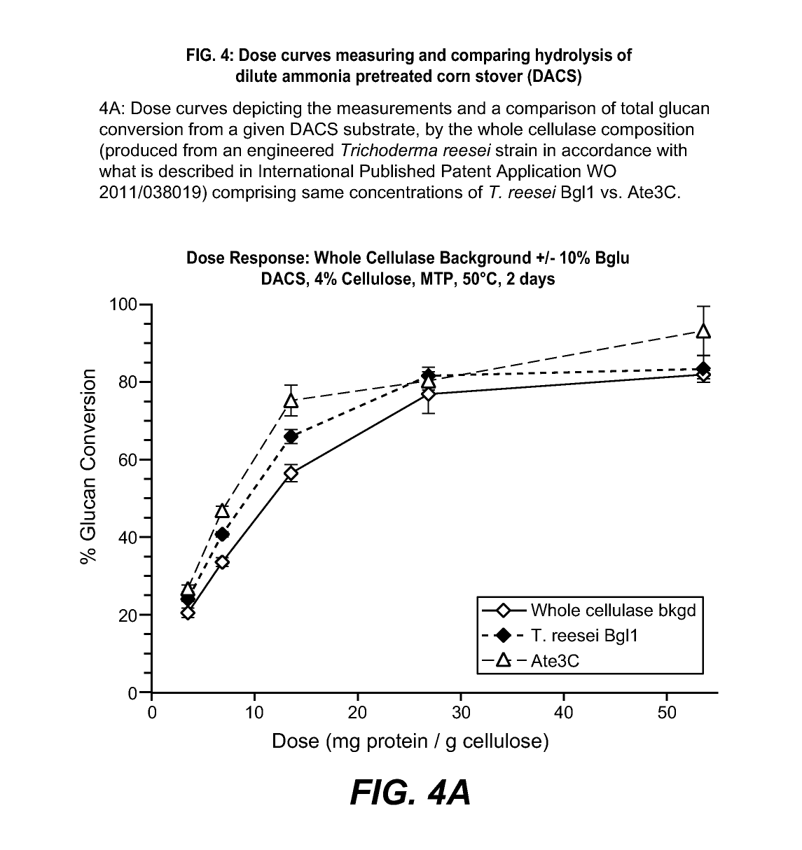

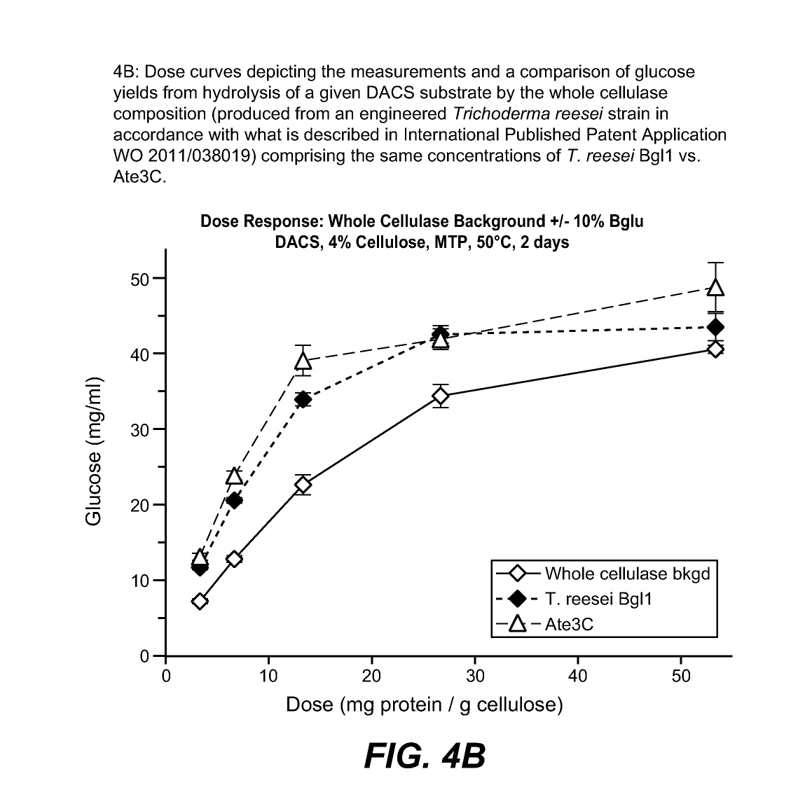

The results are shown in FIG. 4 (including the dose curves of 4A and 4B)

The mixture containing beta-glucosidase with the greater cellobiase activity yielded the most glucan conversion, particularly at doses below 13 mg/g. It is noted that blending the same whole cellulase composition with Ate3C led to greater glucose release and higher overall glucan conversion than the whole cellulase alone or when it is blended with a Trichoderma Bgl1.

5-B: Dose Curves Depicting the Measurements and Comparison of Ate3C Vs. the Benchmark Trichoderma reesei Bgl1 hydrolysis of DACS, wherein the Ate3C and Bgl1 were Added at Increasing Doses to a Whole Cellulase Composition at 13.4 mg/g Produced by a Strain Described in International Published Patent Application No. WO 2011/038019.

In this experiment, the beta-glucosidases were added in increasing dose to a constant loading of 13.4 mg protein/g of glucan of a whole cellulase produced by an engineered Trichoderma reesei strain in accordance with International Published Patent Application No. WO 2011/038019. The mixtures were used to hydrolyze DACS (4% glucan) for 2 days at 50 ° C. To prepare the mixture, T. reesei Bgl1 was added to the mixture from a purified stock of 2.2 g/L total protein, and the whole cellulase was added to the mixture from an 88.8 g/L total protein stock. Ate3C was added from the 2.89 mg/mL concentrate.

Dilute ammonia pretreated corn stover in microtiter plates was prepared as described above. All enzymes were loaded based on mg protein/g glucan in the substrate. All enzyme dilutions were made into 50 mM Sodium Acetate buffer, pH 5.0. Thirty (30) μL of enzyme solution was added to 45 μg substrate per well in microtiter plates. The plates were covered with foil seals and incubated for 2 days at 50° C., 200 rpm in an Innova incubator/shaker. The reaction was quenched with 100 μL of 100 mM Glycine, pH 10, filtered and the soluble sugars measured by HPLC (Agilent 100 series equipped with a de-ashing column (Biorad 125-0118) and carbohydrate column (Aminex HPX-87P). The mobile phase was water with a flow rate of 0.6 ml/min and 20 min run time. A glucose standard curve was generated and used for quantitation.

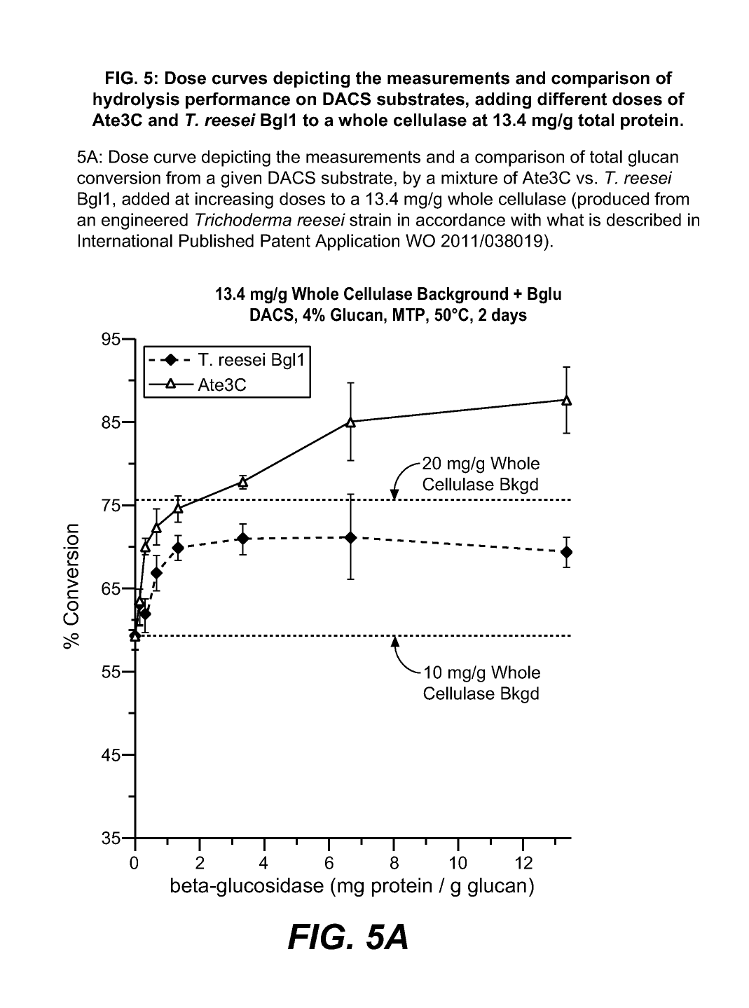

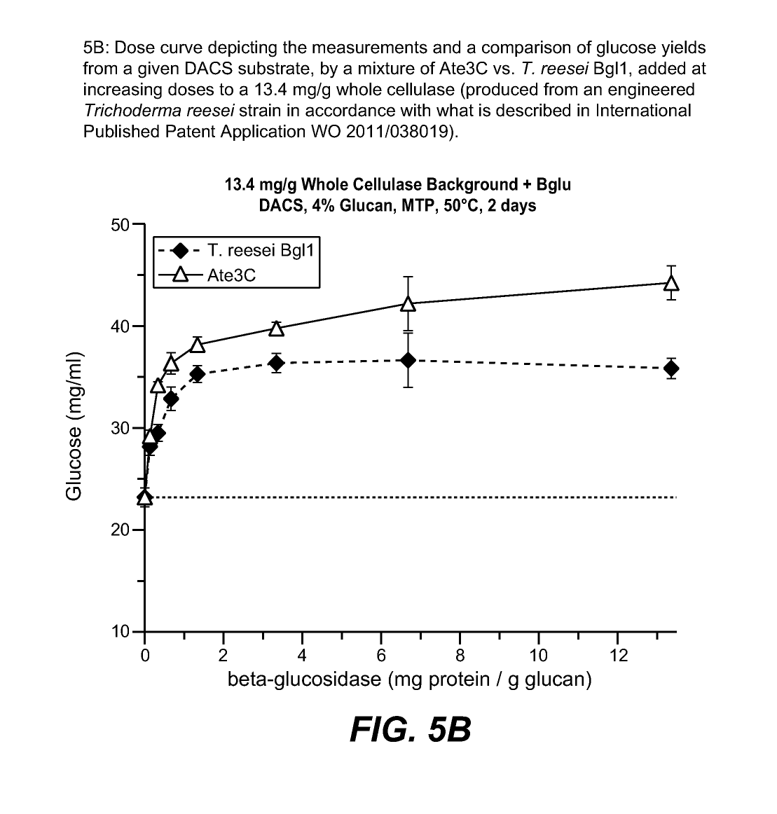

Percent glucan conversion is defined as (mg glucose+mg cellobiose+mg cellotriose)/mg cellulose in the substrate. The results are shown in FIG. 5 (including the dose curves of 5A and 5B).

Ate3C out performed T. reesei Bgl1 at all doses.

We claim:

1. A recombinant polypeptide comprising an amino acid sequence that is at least 85% identical to the amino acid sequence of SEQ ID NO:2 or SEQ ID NO:3, wherein the polypeptide has beta-glucosidase activity.

2. The recombinant polypeptide of claim 1, wherein the polypeptide has improved beta-glucosidase activity as compared to Trichoderma reesei Bgl1 when the recombinant polypeptide and the Trichoderma reesei Bgl1 are used to hydrolyze lignocellulosic biomass substrates.

3. The recombinant polypeptide of claim 1 or 2, wherein the improved beta-glucosidase activity is an increased cellobiase activity.

4. The recombinant polypeptide of any one of claims 1-3, wherein the improved beta-glucosidase activity is an increased yield of glucose and an equal or lower yield of total sugars from a lignocellulosic biomass under the same saccharification conditions.

5. The recombinant polypeptide of claim 4, wherein the lignocellulosic biomass is subject to a pretreatment prior to saccharification.

6. The recombinant polypeptide of any one of claims 1-5, wherein the polypeptide comprises an amino acid sequence that is at least 90% identical to the amino acid sequence of SEQ ID NO:2 or SEQ ID NO:3.

7. The recombinant polypeptide of any one of claims 1-5, wherein the polypeptide comprises an amino acid sequence that is at least 95% identical to the amino acid sequence of SEQ ID NO:2 or SEQ ID NO:3.

8. A composition comprising the recombinant polypeptide of any one of claims 1-7, further comprising one or more other cellulases.

9. The composition of claim 8 wherein the one or more other cellulases are selected from no or one or more other beta-glucosidases, one or more cellobiohydrolases, and one or more endoglucanases.

10. A composition comprising the recombinant polypeptides of any one of claims 1-7, further comprising one or more hemicellulases.

11. The composition of claim 8 or 9, further comprising one or more hemicellulases.

12. The composition of claim 10 or 11, wherein the one or more hemicellulases are selected from one or more xylanases, one or more beta-xylosidases, and one or more L-arabinofuranosidases.

13. A nucleic acid encoding the recombinant polypeptide of any one of claims 1-7.

14. The nucleic acid of claim 13, wherein the polypeptide further comprises a signal peptide sequence.

15. The nucleic acid of claim 14, wherein the signal peptide sequence is selected from the group consisting of SEQ ID NOs: 13-42.

16. An expression vector comprising the nucleic acid of any one of claims 13-15 in operable combination with a regulatory sequence.

17. A host cell comprising the expression vector of claim 16.

18. The host cell of claim 17, wherein the host cell is a bacterial cell or a fungal cell.

19. A composition comprising the host cell of claim 17 or 18 and a culture medium.

20. A method of producing a beta-glucosidase, comprising: culturing the host cell of claim 17 or 18 in a culture medium, under suitable conditions to produce the beta-glucosidase.

21. A composition comprising the beta-glucosidase produced in accordance with the method of claim 20 in supernatant of the culture medium.

22. A method for hydrolyzing a lignocellulosic biomass substrate, comprising:
contacting the lignocellulosic biomass substrate with the polypeptide of any one of claims 1-7, or the composition of claim 21, to yield a glucose and other sugars.
